(19) United States
(12) Patent Application Publication
(10) Pub. No.: US 2025/0011798 A1
(43) Pub. Date: Jan. 9, 2025

(54) MARKERLESS DNA PRODUCTION

(71) Applicant: ModernaTX, Inc., Cambridge, MA (US)

(72) Inventors: Kevin Smith, Cambridge, MA (US); Joe R. Sarkis, Cambridge, MA (US); Stoil Dimitrov, Cambridge, MA (US)

(73) Assignee: ModernaTX, Inc., Cambridge, MA (US)

(21) Appl. No.: 18/702,284

(22) PCT Filed: Oct. 17, 2022
(86) PCT No.: PCT/US2022/078206

§ 371 Date: Apr. 17, 2024

Related U.S. Application Data
(60) Provisional application No. 63/257,054, filed on Oct. 18, 2021.
(60) Provisional application No. 63/346,414, filed on May 27, 2022.

(51) Int. Cl. C12N 15/70; C12P 19/34

(52) U.S. Cl. CPC C12N 15/70; C12P 19/34

(57) ABSTRACT

Provided herein are genetically modified microorganisms comprising a nonsense mutation in a gene encoding an efflux pump or import protein, and a vector encoding a suppressor tRNA that enables translation of the efflux pump or import protein, mitigating the effect of the nonsense mutation. Also provided are methods of producing a vector or markerless DNA using the genetically modified microorganisms provided herein.

### RELATED APPLICATIONS

This application claims the benefit under 35 U.S.C. § 119(e) of the earlier filing dates of U.S. Provisional Application No. 63/257,054, filed Oct. 18, 2021, and U.S. Provisional Application No. 63/346,414, filed May 27, 2022, the contents of which are incorporated by reference herein in their entirety.

### REFERENCE TO AN ELECTRONIC SEQUENCE LISTING

The contents of the electronic sequence listing (M137870167W000-SEQ-NTJ.xml; Size: 34,210 bytes; and Date of Creation: Oct. 13, 2022) are herein incorporated by reference in their entirety.

### BACKGROUND

Successfully delivering DNA to cells for the purpose of gene expression or genome editing requires overcoming three distinct barriers: 1) efficient delivery to the cytoplasm after cellular uptake; 2) avoiding activation of the cellular DNA-activated innate immune response; and 3) nuclear import of DNA through the nuclear envelope. Incorporation of DNA into adeno-associated virus (AAV) particles allows for efficient delivery of the viral genome, and thus the incorporated DNA, into the nucleus of cells. However, only a limited amount of DNA, approximately 4.5 kb, may be incorporated into an AAV particle, and thus AAV-based vectors cannot feasibly deliver larger DNA constructs. Furthermore, the immune response generated to AAV capsid proteins limits the efficiency of DNA delivery by future administration of AAV vectors with the same capsid proteins, preventing therapeutic approaches involving multiple doses. Thus, AAV vector-based approaches are not suitable for delivering large DNA sequences or delivering a DNA construct multiple times.

Microorganisms can be used to replicate DNA vectors, producing large amounts of DNA for delivery to cells, use as a template for in vitro transcription, and other applications. Such DNA vectors often encode antibiotic resistance markers, so that microorganisms can be cultured in the presence of an antibiotic to maintain the presence of the DNA in microbial cells during fermentation. However, antibiotic resistance markers are often large proteins that are burdensome for microorganisms to express. Furthermore, the inclusion of resistance markers increases the total length of the DNA vector, reducing overall yield.

### SUMMARY

Provided herein are engineered bacterial strains and vectors for DNA production without the use of antibiotic resistance genes. DNA production in microorganisms often involves the use of antibiotic resistance markers on the DNA being produced, allowing antibiotics to be added to microbial growth medium for convenient positive selection and maintenance of the DNA in the host microorganism. However, antibiotic resistance markers are energetically costly for bacterial cells to express, and increase the length of the DNA sequence being replicated. These fitness costs and increased DNA lengths reduce the efficiency of DNA production. Additionally, the expression of the antibiotic resistance marker may have unwanted biological effects in downstream applications using the DNA. Alternative methods of positive selection to maintain a DNA template in the host microorganism can thus improve the efficiency of DNA production and avoid undesired expression of antibiotic resistance markers. Introduction of a STOP codon into a gene encoding an efflux pump can prevent a bacterium from expressing a full-length, functional form of the efflux pump, rendering it susceptible to multiple antimicrobial agents, such as nalidixic acid. However, if a bacterial cell expresses a suppressor tRNA, which carries an amino acid and comprises an anticodon complementary to the introduced stop codon, then the introduced STOP codon effectively encodes the amino acid carried by the suppressor tRNA, and the bacterial cell can translate the full-length, functional form of the efflux pump. If the STOP codon is introduced to the genome of a bacterium, and nucleic acid sequence encoding the suppressor tRNA is comprised on a vector, such as a plasmid, then only bacterial cells that contain such a vector will be able to grow in the presence of nalidixic acid. As demonstrated herein, this approach of introducing a STOP codon into a gene encoding an efflux pump, such as tolC, of a bacterium, and introducing a vector with a nucleic acid encoding a suppressor tRNA to a population of bacterial cells, allows for positive selection of bacteria. Alternatively, introduction of a STOP codon into a gene encoding an import protein prevents a bacterium from importing an essential nutrient from the environment, which is lethal for an auxotroph that cannot synthesize the nutrient. Thus, auxotrophs containing nonsense mutations in import proteins will be able to grow if expression of the import proteins is restored by a suppressor tRNA. The bacteria containing the vector may be selected using a variety of compounds toxic to bacteria, such as nalidixic acid. Importantly, this approach allows for the replication of vectors that do not comprise antibiotic resistance markers, which are large, reducing vector copy number, and often metabolically burdensome for bacteria to express. Furthermore, nucleic acid sequences encoding suppressor tRNAs are relatively short, and this approach may thus be used to produce smaller vectors than those that require the use of an antibiotic resistance marker. Reduced vector size and use of a less burdensome element for positive selection allows for more robust microbial growth and increased yield of DNA.

Accordingly, some aspects of the disclosure relate to a genetically modified microorganism comprising
(i) a genome comprising a nonsense mutation in a gene encoding an efflux pump or import protein; and
(ii) a vector comprising a nucleic acid sequence encoding a suppressor tRNA, wherein the nonsense mutation comprises a first STOP codon, and the suppressor tRNA comprises an anticodon that is complementary to the first STOP codon.

In some embodiments, the gene encoding an efflux pump or import protein comprises a second STOP codon that is downstream of the first STOP codon, wherein the second STOP codon comprises a nucleic acid sequence that is not the nucleic acid sequence of the first STOP codon. In some embodiments, the first STOP codon comprises the nucleic acid sequence TAG or UAG. In some embodiments, the first STOP codon comprises the nucleic acid sequence TAA or UAA. In some embodiments, the first STOP codon comprises the nucleic acid sequence TGA or UGA. In some embodiments, the first STOP codon is located in the first 400, first 300, first 250, first 200, first 150, first 100, first 90, first 80, first 70, first 60, first 50, first 40, first 30, first 20, first 10, or first 5 codons of an open reading frame in the gene encoding the efflux pump or import protein. In some embodiments, the first STOP codon is located in the first 10 codons of an open reading frame in the gene encoding the efflux pump or import protein.

In some embodiments, the suppressor tRNA is a histidine tRNA.

In some embodiments, the genome comprises a nucleic acid sequence encoding pir. In some embodiments, a first promoter is operably linked to the nucleic acid sequence encoding pir. In some embodiments, the first promoter is selected from the group consisting of a Kan promoter, LacIq promoter, tre promoter, Lpp promoter, and J23107 promoter. In some embodiments, the first promoter comprises a nucleic acid sequence with at least 90% sequence identity to the nucleic acid sequence of any one of SEQ ID NOs: 3-7.

In some embodiments, the vector comprises an R6Ky origin of replication.

In some embodiments, the vector comprises fewer than 1,000, fewer than 900, fewer than 800, fewer than 700, fewer than 600, or fewer than 500 nucleotides.

In some embodiments, the genome is an E. coli genome.

In some embodiments, the genome comprises a nonsense mutation in a gene encoding an efflux pump, wherein the gene encoding an efflux pump is acrAB, acrD, acrEF, emrD, emrE, emrKY, mdfA, tehAB, tolC, ybhGFSR, ybjY, ybjZ, yegM, yegNO, yhiUV, yjcP, ylcB, or yohG. In some embodiments, the gene encoding an efflux pump is tolC. In some embodiments, the microorganism is capable of growing in the presence of a selective agent. In some embodiments, the selective agent is ampicillin, chloramphenicol, florfenicol, clotrimazole, puromycin, erythromycin, methotrexate, novobiocin, ciprofloxacin, norfloxacin, nalidixic acid, rifampin, fusidic acid, streptomycin, sulfacetamide, tetracycline, deoxycholate, sodium cholate, sodium taurodeoxycholate, sodium oxalate, proflavine, crystal violet, acriflavin, ethidium bromide, tetraphenylphosphonium, rhodamine 6G, tetraphenylarsonium chloride, dequalinium chloride, benzalkonium chloride, daunomycin, plumbagin, or methyl viologen. In some embodiments, the selective agent is nalidixic acid. In some embodiments, the selective agent is deoxycholate.

In some embodiments, the genome comprises a nonsense mutation in a gene encoding an import protein. In some embodiments, the import protein is selected from the group consisting of mppA and oppBCDF. In some embodiments, the microorganism is an auxotroph, wherein the auxotroph is not capable of synthesizing one or more nutrients. In some embodiments, the auxotroph is not capable of synthesizing one or more amino acids.

In some aspects, the disclosure relates to a method of enriching a population of microorganisms for any of the genetically modified microorganisms provided herein, the method comprising exposing a population of microorganisms to a selective agent, wherein the frequency of the genetically modified microorganism in the population is increased after exposure to the selective agent. In some embodiments, the selective agent is ampicillin, chloramphenicol, florfenicol, clotrimazole, puromycin, erythromycin, methotrexate, novobiocin, ciprofloxacin, norfloxacin, nalidixic acid, rifampin, fusidic acid, streptomycin, sulfacetamide, tetracycline, deoxycholate, sodium cholate, sodium taurodeoxycholate, sodium oxalate, proflavine, crystal violet, acriflavine, ethidium bromide, tetraphenylphosphonium, rhodamine 6G, tetraphenylarsonium chloride, dequalinium chloride, benzalkonium chloride, daunomycin, plumbagin, or methyl viologen. In some embodiments, the selective agent is nalidixic acid. In some embodiments, the selective agent is deoxycholate.

In some aspects, the disclosure relates to a method of enriching a population of microorganisms for any of the genetically modified microorganisms provided herein, the method comprising growing a population of microorganisms in the presence of a nutrient, wherein the frequency of the genetically modified microorganism in the population is increased after growth in the presence of the nutrient. In some embodiments, the nutrient is an amino acid.

In some aspects, the disclosure relates to a method of producing a markerless DNA, the method comprising
(i) culturing any of the genetically modified microorganisms provided herein under conditions suitable for replication of the vector; and
(ii) isolating the vector from the microorganism to obtain a markerless DNA.

In some aspects, the disclosure relates to a markerless DNA produced by any of the methods provided herein. In some embodiments, the markerless DNA further comprises an open reading frame encoding a protein. In some embodiments, the markerless DNA further comprises a second promoter operably linked to the open reading frame encoding the protein. In some embodiments, the open reading frame is codon optimized for expression in a cell. In some embodiments, the open reading frame is codon optimized for expression in a human cell.

In some aspects, the disclosure relates to a markerless DNA comprising:
(i) an origin of replication;
(ii) a nucleic acid sequence encoding a suppressor tRNA; and
(iii) a nucleic acid sequence with at least 90% sequence identity to SEQ ID NO: 19.
wherein the suppressor tRNA comprises an anticodon that is complementary to a STOP codon. In some embodiments, the origin of replication comprises fewer than 600, fewer than 500, fewer than 400, or fewer than 350 nucleotides. In some embodiments, the origin of replication is an R6Kγ origin of replication. In some embodiments, the STOP codon comprises the nucleic acid sequence TAG or UAG. In some embodiments, the STOP codon comprises the nucleic acid sequence TAA or UAA. In some embodiments, the STOP codon comprises the nucleic acid sequence TGA or UGA. In some embodiments, the suppressor tRNA is a histidine tRNA.

In some embodiments, the markerless DNA further comprises an open reading frame encoding a protein. In some embodiments, the markerless DNA further comprises a promoter that is operably linked to the open reading frame encoding the protein. In some embodiments, the open reading frame is codon optimized for expression in a cell. In some embodiments, the open reading frame is codon optimized for expression in a human cell. In some embodiments, the markerless DNA encodes an mRNA.

In some aspects, the disclosure relates to a composition comprising any of the markerless DNAs provided herein, formulated in a lipid nanoparticle. In some embodiments, the lipid nanoparticle comprises an ionizable lipid, a neutral lipid, a sterol, and a polyethylene glycol (PEG)-modified lipid. In some embodiments, the lipid nanoparticle comprises: 40-55 mol % ionizable amino lipid; 5-15 mol % non-cationic lipid; 35-45 mol % sterol; and 1-5 mol % PEG-modified lipid.

In some aspects, the disclosure relates to a pharmaceutical composition comprising any of the markerless DNAs or compositions provided herein. In some embodiments, the pharmaceutical composition further comprises a pharmaceutically acceptable excipient.

In some aspects, the disclosure relates to a method comprising administering any of the markerless DNAs, compositions, or pharmaceutical compositions provided herein to a subject in need thereof.

In some aspects, the disclosure relates to a method for delivering a markerless DNA to a specific cell type in a subject in need thereof, the method comprising administering any of the markerless DNAs, compositions, or pharmaceutical compositions provided herein to a subject in need thereof.

In some aspects, the disclosure relates to a method of treating a disease or condition in a subject in need thereof, the method comprising administering any of the markerless DNAs, compositions, or pharmaceutical compositions provided herein to a subject in need thereof.

In some embodiments, the subject is a mammal. In some embodiments, the subject is a human.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows transformation efficiency and plasmid tolerance of Strain 4 strains modified with pir expression cassettes controlled by promoters of varying strengths.

FIG. 2 shows a markerless DNA production strategy, utilizing an E. coli strain with an amber mutation in tolC and an eDNA vector encoding an amber suppressor tRNA. A custom strain of E. coli is generated that possesses a tolC ‘TAG’ amber mutation in place of a codon that encodes a histidine. The tolC mutant is hypersensitive to low concentrations of nalidixic acid, and therefore relies on the presence of plasmid-borne expression of modified tRNA (HisR*) that allows for translation of full-length TolC protein. SEQ ID NO: 20, corresponding to the nucleotide sequence of the suppressor tRNA, is shown.

FIGS. 3A-3D show the use of amber mutations, and use of amber suppressor tRNAs, for markerless episomal DNA production. FIG. 3A shows the introduction of an amber STOP codon in the tolC coding sequence, replacing the codon encoding residue K3 of TolC. An alternative strain was designed in which the codon encoding residue H342 of TolC was replaced with an amber STOP codon. SEQ ID NO: 21 shows the DNA sequence of the open reading frame. SEQ ID NO: 22 shows the amino acid sequence that is not translated due to the introduction of the STOP codon, but is translated when an amber suppressor tRNA is present in the cell. FIG. 3B shows growth of both strains in Terrific Broth media containing kanamycin, after each strain was transformed with a plasmid encoding either i) an amber suppressor tRNA that carries histidine, or ii) a control plasmid encoding luciferase. FIG. 3C shows growth of both strains in Terrific Broth media containing deoxycholate, after each strain was transformed with a plasmid encoding either i) an amber suppressor tRNA that carries histidine, or ii) a control plasmid encoding luciferase. FIG. 3D shows the growth of each strain in media containing kanamycin or deoxycholate, after transformation with a plasmid encoding either i) an amber suppressor tRNA that carries histidine, or ii) a control plasmid encoding luciferase.

FIG. 4 shows the process of i) developing a novel strain with an introduced STOP codon in tolC, but that can support replication of plasmids with R6K origins of replication, ii) developing a plasmid with a R6K origin of replication and encoding an amber suppressor tRNA, and iii) introducing the plasmid of ii) into the strain of i) to produce a system for the production of markerless DNA.

FIGS. 5A-5C show results of fermentation in which E. coli strains were fermented to produce plasmids encoding (i) luciferase and an amber suppressor tRNA carrying histidine (hisR), (ii) luciferase and a kanamycin resistance marker, or (iii) a kanamycin resistance marker alone. FIG. 5A shows the amount of each plasmid DNA (mg DNA per L medium) produced over time. FIG. 5B shows the biomass (g of wet cell weight per L medium) of bacterial strains carrying each plasmid. FIG. 5C shows the productivity (mg of plasmid DNA per g of wet cell weight) of each plasmid over time.

### DETAILED DESCRIPTION

Nonsense Mutations and Suppressor tRNAs

Some aspects of the disclosure relate to genetically modified microorganisms in which the genome comprises a nonsense mutation in a gene encoding an efflux pump or import protein. A “gene encoding a protein,” as used herein, refers to a nucleic acid sequence comprising a coding sequence, or open reading frame, that leads to the production of the protein when the gene is expressed. The nucleic acid sequence may be a DNA sequence, in which case the protein is produced when an RNA polymerase transcribes uses the DNA sequence to transcribe an RNA molecule comprising an RNA sequence that is complementary to the DNA sequence, and translation of the RNA sequence produces a polypeptide with the amino acid sequence of the protein. The nucleic acid sequence may be an RNA sequence, in which case translation of the RNA sequence produces a polypeptide with the amino acid sequence of the protein. A nonsense mutation in a gene refers to a mutation that introduces an in-frame STOP codon between the START and STOP codons of the coding sequence of the gene. The coding sequence of a gene typically begins with a START codon, such as ATG in the DNA sequence (AUG in the RNA sequence), and ends with a STOP codon, such as TAG, TAA, or TGA in the DNA sequence (UAG, UAA, or UGA in the RNA sequence), with the number of bases between the G of the START codon and the T or U of the STOP codon being a multiple of 3 (e.g., 3, 6, 9). If the number of bases between the G of the START codon and the T or U of the introduced STOP codon is a multiple of 3, then the introduced STOP codon is said to be in-frame.

Expression of a gene begins with transcription, in which an RNA polymerase transcribes a DNA template into an RNA molecule, which may be translated into a polypeptide or protein, or modified by one or more processing steps, such as capping, polyadenylation, and/or splicing before being translated. An RNA molecule that can be translated is referred to as a messenger RNA, or mRNA. A DNA or RNA sequence encodes a gene through codons. A codon refers to a group of three nucleotides within a nucleic acid, such as DNA or RNA, sequence. An anticodon refers to a group of three nucleotides within a nucleic acid, such as a transfer RNA (tRNA), that are complementary to a codon, such that the codon of a first nucleic acid associates with the anticodon of a second nucleic acid through hydrogen bonding between the bases of the codon and anticodon. For example, the codon 5′-AUG-3′ on an mRNA has the corresponding anticodon 3′-UAC-5′ on a tRNA. During translation, a tRNA with an anticodon complementary to the codon to be translated associates with the codon on the mRNA, generally to deliver an amino acid that corresponds to the codon to be translated, or to facilitate termination of translation and release of a translated polypeptide from a ribosome.

Translation is the process in which the RNA coding sequence is used to direct the production of a polypeptide. The first step in translation is initiation, in which a ribosome associates with an mRNA, and a first transfer RNA (tRNA) carrying a first amino acid associates with the first codon, or START codon. The next phase of translation, elongation, involves three steps. First, a second tRNA with an anticodon that is complementary to codon following the START codon, or second codon, and carrying a second amino acid, associates with the mRNA. Second, the carbon atom of terminal, non-side chain carboxylic acid moiety of the first amino acid reacts with the nitrogen of the terminal, non-side chain amino moiety of the second amino acid carried, forming a peptide bond between the two amino acids, with the second amino acid being bound to the second tRNA, and the first amino acid bound to the second amino acid, but not the first tRNA. Third, the first tRNA dissociates from the mRNA, and the ribosome advances along the mRNA, such that the position at which the first tRNA associated with the ribosome is now occupied by the second tRNA, and the position previously occupied by the second tRNA is now free for an additional tRNA carrying an additional amino acid to associate with the mRNA. These three steps of 1) association of a tRNA carrying amino acid, 2) formation of a peptide bond, which adds an additional amino acid to a growing polypeptide, and 3) advancement of the ribosome along the mRNA, continue until the ribosome reaches a STOP codon, which results in termination of translation. Generally, tRNAs that associate with STOP codons do not carry an amino acid, so the association of a tRNA that does not carry an amino acid during the elongation step results in cleavage of the bond between the polypeptide and the tRNA carrying the final amino acid in the polypeptide, such that the polypeptide is released from the ribosome.

Translation of a gene in which an in-frame STOP codon has been introduced terminates earlier than translation of an unmutated form of the gene, resulting in the formation of a shorter polypeptide. This shorter polypeptide may have impaired function, or no function, relative to the polypeptide encoded by the unmutated form of the gene.

A suppressor tRNA refers to a tRNA that suppresses the effect of a nonsense mutation, such as those described herein, by preventing the introduced STOP codon from terminating translation. Translation terminates at a STOP codon because a tRNA that associates with the STOP codon does not carry an amino acid, which results in the release of the polypeptide from the ribosome. However, if the tRNA that associates with the introduced STOP codon carries an amino acid, then elongation may proceed, and the introduction will result in incorporation of the amino acid carried by the tRNA into the polypeptide, rather than termination. If the introduced STOP codon has a different sequence than the STOP codon at the end of the coding sequence of the unmodified form of the gene, then the presence of the suppressor tRNA in the cell will allow elongation to proceed despite the introduced STOP codon, but not affect termination, as the mRNA will still be bound by a different tRNA that does not carry an amino acid.

The genetic code, or collection of codons and their corresponding tRNAs or amino acids, contains three conventional STOP codons: amber, ochre, and opal (alternatively “umber”). In some embodiments of the nonsense mutations provided herein, the introduced STOP codon is an amber STOP codon. An amber STOP codon comprises the DNA sequence TAG or RNA sequence UAG. In some embodiments, the suppressor tRNA is an amber suppressor tRNA. An amber suppressor tRNA comprises the anticodon AUC. In some embodiments, the STOP codon is an ochre STOP codon. An ochre STOP codon comprises the DNA sequence TAA or RNA sequence UAA. In some embodiments, the suppressor tRNA is an ochre suppressor tRNA. An opal suppressor comprises the anticodon AUU. In some embodiments, the STOP codon is an opal or umber STOP codon. An opal or umber STOP codon comprises the DNA sequence TGA or RNA sequence UGA. In some embodiments, the suppressor tRNA is an opal suppressor tRNA or an umber suppressor tRNA. Opal and amber suppressor tRNAs comprise the anticodon ACU.

A first STOP codon may be introduced into an open reading frame of a gene at any in-frame position (i.e., separated from the START codon by 0, 3, 6, 9, or any multiple of 3 nucleotides) downstream of the START codon. Translation initiates at the START codon and proceeds until the STOP codon is reached, when translation terminates. Introduction of a STOP codon closer to the START codon results in translation of a shorter protein fragment. Longer protein fragments may retain some functionality, or localization sequences that result in the translated fragment being retained in the cell, or exported to the outer membrane in the case of an efflux pump or import protein. Introducing a first STOP codon closer to the START codon of an open reading frame thus reduces the potential of translated protein fragments to interfere with other biological processes in the cell, while still allowing suppressor tRNAs to promote translation of the full-length protein. In some embodiments, the first STOP codon is located in the first 400, first 300, first 250, first 200, first 150, first 100, first 90, first 80, first 70, first 60, first 50, first 40, first 30, first 20, first 10, or first 5 codons of an open reading frame in the gene encoding the efflux pump or import protein. In some embodiments, the first STOP codon is located in the first 100 codons of an open reading frame in the gene encoding the efflux pump or import protein. In some embodiments, the first STOP codon is located in the first 50 codons of an open reading frame in the gene encoding the efflux pump or import protein. In some embodiments, the first STOP codon is located in the first 25 codons of an open reading frame in the gene encoding the efflux pump or import protein. In some embodiments, the first STOP codon is located in the first 10 codons of an open reading frame in the gene encoding the efflux pump or import protein. In some embodiments, the first STOP codon is located in the first 5 codons of an open reading frame in the gene encoding the efflux pump or import protein.

In some embodiments, the suppressor tRNA is a histidine tRNA. A histidine tRNA is a tRNA that carries the amino acid histidine. tRNAs are loaded with amino acids by cellular enzymes, such as synthetases, based on their RNA sequence, such that a tRNA of a particular sequence is specifically loaded with a particular amino acid. Loading refers to the process by which an amino acid is covalently bonded to a tRNA, forming an aminoacyl-tRNA, or a tRNA carrying an amino acid. Different suppressor tRNAs comprising the same anticodon but different RNA sequences may thus carry different amino acids, based on their relative ability to be loaded with certain amino acids by cellular enzymes such as synthetases. A representative histidine suppressor tRNA sequence is given by SEQ ID NO: 13. In some embodiments, the suppressor tRNA is an amber suppressor tRNA. In some embodiments, the amber suppressor tRNA comprises the nucleic acid sequence of SEQ ID NO: 13. In some embodiments, the suppressor tRNA is an ochre suppressor tRNA. In some embodiments, the suppressor tRNA comprises the nucleic acid sequence of SEQ ID NO: 14. In some embodiments, the suppressor tRNA is an opal suppressor tRNA. In some embodiments, the suppressor tRNA comprises the nucleic acid sequence of SEQ ID NO: 15.

### Efflux Pumps, Import Proteins, and Selection

Some aspects of the disclosure relate to genetically modified microorganisms comprising a nonsense mutation in a gene encoding an efflux pump. An efflux pump, or efflux transporter, is a protein involved in the transfer of potentially toxic substrates from the inside of a cell into the extracellular environment. Genes encoding efflux pumps are common in the genomes of microorganisms, with many microorganisms producing multiple efflux pumps. Typically, efflux pumps are embedding in the cell membrane and/or cell wall of a microorganism and, by exporting toxic substances from the interior of the cell, prevent these substances from interfering with cellular metabolism and other functions. Thus, the action of efflux pumps confers some degree of resistance to the effects of many toxic substances, such as antibiotics. Conversely, a microorganism with reduced efflux pump activity, such as through loss of expression of one or more efflux pumps, can be more sensitive to the action of toxic substances, such as antibiotics (see, e.g., Webber et al. J Antimicrob Chemother. 2003. 51(1):9-11).

Efflux pumps are typically classified as belonging to one or more of the following classes: resistance-nodulation-cell division (RND), major facilitator (MF), small multidrug resistance (SMR), ATP-binding cassette (ABC), or multidrug and toxic efflux (MATE) (see, e.g., Amaral et al. Front Pharmacol. 2013. 4:168).

RND efflux pumps operate as part of a tripartite complex including an RND efflux pump in the inner membrane, an adaptor MF efflux pump located in the periplasm between the inner and outer membranes, and an outer membrane protein (OMP) located in the outer membrane. RND efflux pumps export a broad spectrum of compounds, including heavy metals, hydrophobic, and amphiphilic compounds from the cytoplasm into the periplasmic space. After entering the periplasmic space, compounds are exported by an MF efflux pump (see, e.g. Kumar et al. Int J Mol Sci. 2012. 13(4):4484-4495). Finally, an OMP exports the substance into the extracellular environment. SMR efflux pumps, like RND and MF efflux pumps, are driven by proton motive force, and thus depend on a pH gradient between the cell membrane, cell wall, or outer membrane, while MATE efflux pumps utilize Na⁺ or H⁺ antiport mechanisms to export substances for the extrusion of compounds (see, e.g., Delmar et al. Annu Rev Biophys. 2016. 43:97-117). ABC efflux pumps use ATP as an energy source to extrude toxic compounds form the cell. Upon hydrolyzation of ATP, the ABC efflux pump undergoes a conformational change that facilitates the extrusion of a compound from the cytoplasm to the exterior of the plasma membrane.

Non-limiting examples of efflux pumps include those encoded by the genes acrAB, acrD, acrEF, emrD, emrE, emrKY, mdfA, tehAB, tolC, ybhGFSR, ybjY, ybjZ, yegM, yegNO, yhiUV, yjcP, ylcB, and yohG. Non-limiting examples of the substances with toxic activity that may be mitigated by the activity of efflux pumps include ampicillin, chloramphenicol, florfenicol, clotrimazole, puromycin, erythromycin, methotrexate, novobiocin, ciprofloxacin, norfloxacin, nalidixic acid, rifampin, fusidic acid, streptomycin, sulfacetamide, tetracycline, deoxycholate, sodium cholate, sodium taurodeoxycholate, sodium oxalate, proflavine, crystal violet, acriflavin, ethidium bromide, tetraphenylphosphonium, rhodamine 6G, tetraphenylarsonium chloride, dequalinium chloride, benzalkonium chloride, daunomycin, plumbagin, and methyl viologen.

Some aspects of the disclosure relate to genetically modified microorganisms comprising a genome comprising a nonsense mutation in a gene encoding an efflux pump; and a vector comprising a nucleic acid sequence encoding a suppressor tRNA with an anticodon that is complementary to the STOP codon introduced by the nonsense mutation. Expression of the suppressor tRNA allows the microorganism to express the efflux pump, rather than the truncated form that would be expressed in the absence of the suppressor tRNA. Thus, the genetically modified microorganism with a genome comprising the nonsense mutation and expressing the suppressor tRNA is capable of growing in the presence of a selective agent. A selective agent is a substance, such as a protein, lipid, carbohydrate, antibiotic, or small molecule that inhibits one or more biological processes of a microorganism. A selective agent may be used to selectively kill, or inhibit the growth of, microorganisms with genomes comprising the nonsense mutation in a gene encoding an efflux pump that do not express the suppressor tRNA, as they are less able to export toxic substances from the intracellular environment.

In some embodiments, the genetically modified microorganism is capable of growing in the presence of a selective agent. If a microorganism is observed to replicate in an environment containing the selective agent, the microorganism is said to be capable of growing in the presence of the selective agent. Non-limiting examples of environments containing a selective agent include liquid medium, such as LB broth, or solid agar, such as LB agar, in which the selective agent is dissolved. Methods of determining whether a microorganism is capable of growing in the presence of a selective are known in the art. For example, the same microorganism may be introduced into separate tubes containing the same liquid medium (e.g. LB broth), wherein each tube contains a different concentration of the selective agent, or no selective agent. Tubes may then be incubated under conditions suitable for replication of the microorganism (e.g., 37° C.) for a given period of time (e.g., 12 hours), with the number of microorganisms present in each tube being monitored over the period of time, and/or at the end of the incubation. The growth rate of the microorganism, and/or the final population size of the microorganism, in each tube may then be calculated. The microorganism is said to be capable of growing in the presence of the selective agent if the growth rate and/or final population size of the microorganism, when grown in the presence of the selective agent, is at least 10%, at least 20%, at least 30%, at least 40%, at least 50%, at least 60%, at least 70%, at least 80%, at least 90%, at least 95%, at least 96%, at least 97%, at least 98%, at least 99%, or at least 100% of the growth rate of the microorganism grown in the absence of the selective agent.

Some aspects of the disclosure relate to a method of enriching a population of microorganisms for one of the genetically modified microorganism provided herein, comprising exposing a population of microorganisms to a selective agent, wherein the frequency of the genetically modified microorganism in the population is increased after exposure to the selective agent. The frequency of genetically modified microorganisms may be determined by a number of methods that are known in the art. For example, the number of genetically modified microorganisms present in the population may be estimated by obtaining a sample from a medium or composition comprising the population, and counting the number of colony-forming units (CFUs) after introducing the sample to agar plates containing the selective agent, such that only genetically modified microorganisms form colonies on the agar. Then, the total number of microorganisms present in the population may be quantified by counting the number of colony-forming units after the sample is introduced to agar plates that do not comprise the selective agent. The frequency may then be calculated by dividing the number of genetically modified microorganisms in the population by the total number of genetically modified microorganisms in the population. Determining whether exposure to a selective agent increases the frequency of the genetically modified microorganism in the population may be achieved by measuring the frequency before exposure, introducing the selective agent to a medium or composition comprising the population, incubating the population with the selective agent for a given period of time, and measuring the frequency after the exposure or incubation.

In some embodiments, the selective agent is ampicillin, chloramphenicol, florfenicol, clotrimazole, puromycin, erythromycin, methotrexate, novobiocin, ciprofloxacin, norfloxacin, nalidixic acid, rifampin, fusidic acid, streptomycin, sulfacetamide, tetracycline, deoxycholate, sodium cholate, sodium taurodeoxycholate, sodium oxalate, proflavine, crystal violet, acriflavin, ethidium bromide, tetraphenylphosphonium, rhodamine 6G, tetraphenylarsonium chloride, dequalinium chloride, benzalkonium chloride, daunomycin, plumbagin, or methyl viologen. In some embodiments, the selective agent is nalidixic acid. Nalidixic acid is a quinolone compound with potent antimicrobial activity. Quinolones, such as nalidxic acid, bind to DNA gyrase-DNA complex, which inhibits DNA replication and thereby prevents bacterial replication. Additionally, quinolones can inhibit the activities of E. coli topoisomerase (see, e.g. Hooper. Drugs. 1995. 49 Suppl 2:10-15). In some embodiments, the selective agent is deoxycholate.

Some aspects of the disclosure relate to genetically modified microorganisms comprising a nonsense mutation in a gene encoding an import protein. Import proteins are proteins involved in the transfer of molecules such as lipids, carbohydrates, amino acids, and/or peptides from the extracellular environment into the periplasm or cytoplasm of the bacterial cell. For example, murein peptide permease A (MppA) is a periplasmic binding protein that is essential for the import of the peptide L-alanyl-gamma-D-glutamyl-mesodiaminopimelate, and oligopeptide permease (Opp) is involved in the import of small peptides. If a bacterium is an auxotroph that cannot synthesize an essential nutrient, such as proline, the OppBCDF or MppA import proteins allow the bacterium to import the nutrient from the extracellular environment. See, e.g., Park et al. J Bacteriol. 1998. 180(5):1215-1223.

A nonsense mutation that prevents translation of a functional form of the import protein can prevent the bacterium from importing the nutrient from the environment. Inability to import a nutrient from the environment imposes a fitness cost to the bacterium. If the nutrient is essential, and the bacterium is an auxotroph that is unable to synthesize the nutrient itself, then such a nonsense mutation is lethal. However, suppressing the effects of nonsense mutation using a suppressor tRNA allows bacteria to produce functional import proteins and import the required nutrient. Thus, providing a vector encoding the suppressor tRNA to auxotrophic bacteria with a nonsense mutation in an import protein, and culturing the bacteria in the presence of a nutrient that must be imported, allows for positive selection of bacteria that contain the vector. Non-limiting examples of import proteins include mppA and oppBCDF.

In some embodiments, the genetically modified microorganism is an auxotroph that is not capable of synthesizing a nutrient. If a microorganism is observed to replicate in an environment containing the nutrient, but not in an environment that does not contain the nutrient, the microorganism is said to be incapable of synthesizing the nutrient, and auxotrophic with respect to the nutrient. Non-limiting examples of nutrients include amino acids, monosaccharides, and lipids. Methods of determining whether a microorganism is capable of growing in the presence or absence of a nutrient are known in the art. For example, the same microorganism may be introduced into separate tubes containing the same defined medium, for which the exact concentration of individual compounds is known, wherein the nutrient is added to one tube, and one tube does not contain the nutrient. Tubes may then be incubated under conditions suitable for replication of the microorganism (e.g., 37° C.) for a given period of time (e.g., 12 hours), with the number of microorganisms present in each tube being monitored over the period of time, and/or at the end of the incubation. The growth rate of the microorganism, and/or the final population size of the microorganism, in each tube may then be calculated. The microorganism is auxotrophic with respect to the nutrient if the growth rate and/or final population size of the microorganism, when grown in the presence of the nutrient, is at least 10%, at least 20%, at least 30%, at least 40%, at least 50%, at least 60%, at least 70%, at least 80%, at least 90%, at least 95%, at least 96%, at least 97%, at least 98%, at least 99%, or at least 100% higher than that of the microorganism grown in the absence of the nutrient.

Some aspects of the disclosure relate to a method of enriching a population of microorganisms for one of the genetically modified microorganism provided herein, comprising growing a population of microorganisms in the presence of a nutrient, wherein the frequency of the genetically modified microorganism in the population is increased after growth in the presence of the nutrient. The frequency of genetically modified microorganisms may be determined by a number of methods that are known in the art. For example, the number of genetically modified microorganisms present in the population may be estimated by obtaining a sample from a medium or composition comprising the population, and counting the number of colony-forming units (CFUs) after introducing the sample to agar plates containing the nutrient, such that only genetically modified microorganisms form colonies on the agar. Because bacteria lacking the vector cannot import the nutrient, the total number of bacteria in a sample can instead be quantified by a culture-independent method, such as qPCR on a genomic target to measure the number of bacterial genomes. The frequency may then be calculated by dividing the number of genetically modified microorganisms in the population by the total number of bacterial genomes. Determining whether growth in a nutrient increases the frequency of the genetically modified microorganism in the population may be achieved by measuring the frequency before growth, introducing the nutrient to a defined medium or composition comprising the population, incubating the population with the nutrient for a given period of time, and measuring the frequency after the exposure or incubation.

Some aspects of the disclosure relate to a method of producing a markerless DNA, the method comprising culturing any of the genetically modified microorganisms provided herein under conditions suitable for replication of the vector; and isolating the vector from the microorganism to obtain a markerless DNA. Microorganisms may be cultured by any of the methods provided herein, using positive selection to favor replication of microorganisms that contain the vector. Once the microorganism reaches a suitable population size, such as the carrying capacity of the microorganism in the culture environment, microorganism cells are lysed to release the vector into the extracellular space, and the vector is purified from cellular debris. Methods of isolating a vector from a microorganism are known in the art. Generally, bacterial cells are lysed by exposure to an alkaline environment or heating, cellular debris is separated from supernatant by centrifugation and/or filtration, and vector DNA is purified by salt precipitation or a column-based method.

### R6K Origins of Replication and Pir

In some embodiments, the suppressor tRNA is encoded by a vector, such as a plasmid. A plasmid is a circular DNA polynucleotide that is capable of being replicated independently of the chromosome of a cell. An origin of replication (ori) of a DNA polynucleotide refers to a DNA sequence at which replication of the DNA polynucleotide initiates. The origin of replication of a plasmid influences the copy number of the plasmid in a bacterial cell or other cells containing the plasmid. The copy number of a plasmid refers to the number of plasmid molecules per cell. A plasmid with an pUC origin of replication has a copy number of about 500-700 copies per cell, while a plasmid with an R6Kγ origin of replication has a copy number of about 15-20 copies per cell.

In some embodiments, the genome of the genetically modified microorganisms provided herein comprises a nucleic acid sequence encoding the π (Pi) protein. The π protein, encoded by the gene pir, is required for replication of plasmids with an origin of replication derived from the R6K replicon (see, e.g. Rakowski et al. Plasmid. 2013. 69(3):231-242). A representative nucleotide sequence encoding the pir gene is given by SEQ ID NO: 9, and a representative amino acid sequence of the π (Pi) protein is given by SEQ ID NO: 10. In some embodiments, the genome of the microorganism comprises a promoter operably linked to the nucleic acid sequence encoding the pir gene or Pi protein. A promoter is said to be operably linked to a gene if the promoter controls the degree to which the gene is expressed. In some embodiments, the promoter may regulate conditional expression of the open reading frame with which it is operably linked, such that the encoded protein is produced selectively under certain desired conditions, such as the presence or absence of a particular environmental signal, or in a certain cell type. Non-limiting examples of promoters include Kan promoter, LacIq promoter, trc promoter, Lpp promoter, and J23107 promoter. In some embodiments, the promoter is selected from the group consisting of a Kan, LacIq, trc, Lpp, and J23107 promoter. In some embodiments, the promoter comprises a nucleic acid sequence with at least 90% sequence identity to any one of SEQ ID NOs: 3-7. In some embodiments, the promoter is a Kan promoter. In some embodiments, the Kan promoter comprises a nucleic acid sequence with at least 90% sequence identity to the nucleic acid sequence of SEQ ID NO: 4. In some embodiments, the promoter is a LacIq promoter. In some embodiments, the LacIq promoter comprises a nucleic acid sequence with at least 90% sequence identity to the nucleic acid sequence of SEQ ID NO: 5. In some embodiments, the promoter is a trc promoter. In some embodiments, the trc promoter comprises a nucleic acid sequence with at least 90% sequence identity to the nucleic acid sequence of SEQ ID NO:6. In some embodiments, the promoter is a Lpp promoter. In some embodiments, the Lpp promoter comprises a nucleic acid sequence with at least 90% sequence identity to the nucleic acid sequence of SEQ ID NO: 7. In some embodiments, the promoter is a J23107 promoter. In some embodiments, the J23107 promoter comprises a nucleic acid sequence with at least 90% sequence identity to the nucleic acid sequence of SEQ ID NO: 3.

The R6K replicon comprises three distinct origins of replication, R6Kα, R6Kβ, and R6Kγ. Representative nucleotide sequences of the R6Kα, R6Kβ, and R6Kγ origins of replication are given by SEQ ID NOs: 16 (R6Kα), 17 (R6Kβ), and 18 (R6Kγ). The π protein binds to, and initiates replication at, each of the R6Kα, R6Kβ, and R6Kγ origins of replication, but a plasmid can be replicated if it contains any one of these three origins of replication.

In some embodiments, the vector comprises fewer than 1,000, fewer than 900, fewer than 800, fewer than 700, fewer than 600, or fewer than 500 nucleotides. In some embodiments, the origin of replication of the vector comprises fewer than 600, fewer than 500, fewer than 400, or fewer than 350 nucleotides. The smallest of the R6K-derived origins of replication is R6Kγ, which comprises 382 bp. Thus, substitution of a plasmid's origin of replication with an R6Kγ origin of replication tends to reduce the size of the plasmid. Smaller plasmids are replicated faster than larger plasmids, and comprise fewer CpG motifs. A CpG motif is a dinucleotide sequence in a DNA molecule comprising a cytosine followed by a guanine, wherein a phosphate moiety is bonded to the 3′ carbon of the cytosine and 5′ carbon of the guanine. Toll-like receptors (TLRs), such as Toll-like receptor 9 (TLR9), bind to CpG motifs on DNA, and initiate an inflammatory response following binding of the receptor to CpG motif. A vector or plasmid with fewer CpG motifs is thus less likely to induce such an inflammatory response, or induce less inflammation, when present in subject, such as a human subject.

Vectors Encoding Proteins and Suppressor tRNAs

The vectors and markerless DNAs provided herein, in some embodiments, comprise an open reading frame encoding a protein. As used herein, a “markerless DNA” refers to a DNA that does not encode an antibiotic resistance marker. An open reading frame is a continuous stretch of DNA beginning with a START codon (e.g., methionine (ATG)), and ending with a STOP codon (e.g., TAA, TAG or TGA) and encodes a polypeptide. An open reading frame is said to encode a polypeptide if, following transcription of the DNA sequence of the open reading frame, the resulting RNA can be translated into the polypeptide. An open reading frame may comprise a DNA sequence that, when transcribed by an RNA polymerase, can be translated into a polypeptide. Alternatively, an open reading frame may comprise one or more introns, such that when the open reading frame is transcribed to produce an RNA, the RNA must be spliced before it can be translated into the polypeptide. In some embodiments, the vector or markerless DNA encodes an mRNA.

The nucleic acids, for example vectors and markerless DNAs, of the disclosure may be formulated in appropriate carriers or delivery vehicles (e.g., lipid nanoparticles), such that the nucleic acids, e.g., markerless DNAs, are suitable for use in vivo. When appropriately formulated, nucleic acids, e.g., markerless DNAs, are capable of being delivered to cells and/or tissues within a subject, e.g., a human subject, to effectuate translation of protein encoded by these nucleic acids. As used herein, the term “nucleic acid” refers to multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). As used herein, the term nucleic acid refers to polyribonucleotides as well as polydeoxyribonucleotides. The term nucleic acid shall also include polynucleosides (i.e., a polynucleotide minus the phosphate) and any other organic base containing polymer. Non-limiting examples of nucleic acids include chromosomes, genomic loci, genes or gene segments that encode polynucleotides or polypeptides, coding sequences, non-coding sequences (e.g., intron, 5′-UTR, or 3′-UTR) of a gene, pri-mRNA, pre-mRNA, cDNA, mRNA, etc. A nucleic acid may include a substitution and/or modification. In some embodiments, the substitution and/or modification is in one or more bases and/or sugars. For example, in some embodiments a nucleic acid includes nucleic acids having backbone sugars that are covalently attached to low molecular weight organic groups other than a hydroxyl group at the 2′ position and other than a phosphate group or hydroxy group at the 5′ position. Thus, in some embodiments, a substituted or modified nucleic acid includes a 2′-O-alkylated ribose group. In some embodiments, a modified nucleic acid includes sugars such as hexose, 2′-F hexose, 2′-amino ribose, constrained ethyl (cEt), locked nucleic acid (LNA), arabinose or 2′-fluoroarabinose instead of ribose. Thus, in some embodiments, a nucleic acid is heterogeneous in backbone composition thereby containing any possible combination of polymer units linked together such as peptide-nucleic acids (which have an amino acid backbone with nucleic acid bases).

The nucleic acid sequences include nucleic acid sequences that have been removed from their naturally occurring environment, recombinant or cloned DNA isolates, and chemically synthesized analogues or analogues biologically synthesized by heterologous systems.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a bacterial nucleotide sequence, a human nucleotide sequence, and/or a viral nucleotide sequence. Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing. A nucleic may comprise naturally occurring nucleotides and/or non-naturally occurring nucleotides such as modified nucleotides.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

A nucleic acid vector or markerless DNA may include an insert which may be an expression cassette or open reading frame (ORF). An “open reading frame” is a continuous stretch of DNA beginning with a start codon (e.g., methionine (ATG)), and ending with a stop codon (e.g., TAA, TAG or TGA) and encodes a protein or peptide (e.g., a therapeutic protein or therapeutic peptide). In some embodiments, an expression cassette encodes a RNA (e.g., mRNA) including at least the following elements: a 5′ untranslated region, an open reading frame region encoding the mRNA, a 3′ untranslated region and a polyA tail. The open reading frame may encode any mRNA sequence, or portion thereof.

In some embodiments, a nucleic acid vector or markerless DNA encodes an mRNA comprising a 5′ untranslated region (UTR). A “5′ untranslated region (UTR)” refers to a region of an mRNA that is directly upstream (i.e., 5′) from the start codon (i.e., the first codon of an mRNA transcript translated by a ribosome) that does not encode a protein or peptide.

In some embodiments, a nucleic acid vector or markerless DNA encodes an mRNA comprising a 3′ untranslated region (UTR). A “3′ untranslated region (UTR)” refers to a region of an mRNA that is directly downstream (i.e., 3′) from the stop codon (i.e., the codon of an mRNA transcript that signals a termination of translation) that does not encode a protein or peptide.

The terms 5′ and 3′ are used herein to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left-hand side, and 3′ (downstream) indicates genetic elements positioned towards the right-hand side, when following this convention.

A nucleic acid (e.g., DNA or mRNA) typically comprises a plurality of nucleotides. A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally-occurring nucleotides, synthetic nucleotides and modified nucleotides, unless indicated otherwise. Examples of naturally-occurring nucleotides used for the production of RNA, e.g., in an IVT reaction, as provided herein include adenosine triphosphate (ATP), guanosine triphosphate (GTP), cytidine triphosphate (CTP), uridine triphosphate (UTP), and 5-methyluridine triphosphate (m5UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO4 to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir. Modified nucleotides may include modified nucleobases. For example, a RNA transcript (e.g., mRNA transcript) may include a modified nucleobase selected from pseudouridine (ψ), 1-methylpseudouridine (mly), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, a RNA transcript (e.g., mRNA transcript), vector, or markerless DNA includes a combination of at least two (e.g., 2, 3, 4 or more) of the foregoing modified nucleobases.

Some embodiments comprise compositions with at least about 0.25 mg/mL nucleic acid (e.g., DNA), such as 0.5 mg/mL, 0.75 mg/mL, 1 mg/mL, 1.25 mg/mL, 1.5 mg/mL, or 2 mg/mL nucleic acid.

In some embodiments, the vector or markerless DNA comprises a promoter that is operably linked to the open reading frame encoding the protein. A promoter is said to be operably linked to a gene if the promoter controls the degree to which the gene is expressed. In some embodiments, the promoter may regulate conditional expression of the open reading frame with which it is operably linked, such that the encoded protein is produced selectively under certain desired conditions, such as the presence or absence of a particular environmental signal, or in a certain cell type. Non-limiting examples of promoters include Kan promoter, LacIq promoter, tre promoter, Lpp promoter, and J23107 promoter. In some embodiments, the promoter is selected from the group consisting of a Kan, LacIq, tre, Lpp, and J23107 promoter. In some embodiments, the promoter comprises a nucleic acid sequence with at least 90% sequence identity to any one of SEQ ID NOs: 3-7. In some embodiments, the promoter is a Kan promoter. In some embodiments, the Kan promoter comprises a nucleic acid sequence with at least 90% sequence identity to the nucleic acid sequence of SEQ ID NO: 4. In some embodiments, the promoter is a LacIq promoter. In some embodiments, the LacIq promoter comprises a nucleic acid sequence with at least 90% sequence identity to the nucleic acid sequence of SEQ ID NO: 5. In some embodiments, the promoter is a trc promoter. In some embodiments, the tre promoter comprises a nucleic acid sequence with at least 90% sequence identity to the nucleic acid sequence of SEQ ID NO: 6. In some embodiments, the promoter is a Lpp promoter. In some embodiments, the Lpp promoter comprises a nucleic acid sequence with at least 90% sequence identity to the nucleic acid sequence of SEQ ID NO: 7. In some embodiments, the promoter is a J23107 promoter. In some embodiments, the J23107 promoter comprises a nucleic acid sequence with at least 90% sequence identity to the nucleic acid sequence of SEQ ID NO: 3.

In some embodiments, the open read frame encoding the protein is codon optimized for expression in a cell. In some embodiments, the open reading frame encoding the protein is codon optimized for expression in a bacterial cell. In some embodiments, the open reading frame is codon optimized for expression in a human cell. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, the vectors or markerless DNAs provided herein comprise a nucleic acid sequence encoding a suppressor tRNA. In some embodiments, the suppressor tRNA is an amber suppressor tRNA. In some embodiments, the nucleic acid sequence encoding the amber suppressor tRNA comprises the nucleic acid sequence of SEQ ID NO: 13. In some embodiments, the suppressor tRNA is an ochre suppressor tRNA. In some embodiments, the nucleic acid sequence encoding the ochre suppressor tRNA comprises the nucleic acid sequence of SEQ ID NO: 14. In some embodiments, the suppressor tRNA is an opal suppressor tRNA. In some embodiments, the nucleic acid sequence encoding the opal suppressor tRNA comprises the nucleic acid sequence of SEQ ID NO: 15.

In some embodiments, the vector or markerless DNA comprises a promoter operably linked to the nucleic acid sequence encoding the suppressor tRNA. In some embodiments, the promoter is selected from the group consisting of a Kan, LacIq, trc, Lpp, and J23107 promoter. In some embodiments, the promoter comprises a nucleic acid sequence with at least 90% sequence identity to any one of SEQ ID NOs: 3-7. In some embodiments, the promoter is a Kan promoter. In some embodiments, the Kan promoter comprises a nucleic acid sequence with at least 90% sequence identity to the nucleic acid sequence of SEQ ID NO: 4. In some embodiments, the promoter is a LacIq promoter. In some embodiments, the LacIq promoter comprises a nucleic acid sequence with at least 90% sequence identity to the nucleic acid sequence of SEQ ID NO: 5. In some embodiments, the promoter is a trc promoter. In some embodiments, the trc promoter comprises a nucleic acid sequence with at least 90% sequence identity to the nucleic acid sequence of SEQ ID NO: 6. In some embodiments, the promoter is a Lpp promoter. In some embodiments, the Lpp promoter comprises a nucleic acid sequence with at least 90% sequence identity to the nucleic acid sequence of SEQ ID NO: 7. In some embodiments, the promoter is a J23107 promoter. In some embodiments, the J23107 promoter comprises a nucleic acid sequence with at least 90% sequence identity to the nucleic acid sequence of SEQ ID NO: 3.

### Compositions Comprising Vectors or Markerless DNAs

The vectors provided herein may be formulated in a pharmaceutical composition comprising a vector and a pharmaceutically acceptable excipient. The markerless DNAs provided herein may be formulated in a pharmaceutical composition comprising a markerless DNA and a pharmaceutically acceptable excipient. A pharmaceutically acceptable excipient can also be incorporated in a formulation and can be any excipient (e.g., carrier) known in the art. Non-limiting examples include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such excipients.

Pharmaceutically acceptable excipients are known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, and silicylate. Each pharmaceutically acceptable excipients used in a pharmaceutical composition must be “acceptable” in the sense of being compatible with the other ingredients of the formulation and not injurious to the subject. Excipients suitable for a selected dosage form and intended route of administration are well known in the art, and acceptable diluents or carriers for a chosen dosage form and method of administration can be determined using ordinary skill in the art.

In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with DNA or RNA (e.g., for transplantation into a subject), hyaluronidase, nanoparticle mimics and combinations thereof.

Routes of administration of the vectors and markerless DNAs provided herein include, for example, intravenous, intramuscular, intraperitoneal, subcutaneous, or intranasal. Thus, in some embodiments, a composition comprising vector or markerless DNA may be formulated for intravenous, intramuscular, intraperitoneal, subcutaneous, or intranasal delivery.

### Methods of Treatment

Some aspects of the disclosure relate to methods comprising administering a vector, markerless DNA, or pharmaceutical composition provided herein to a subject in need thereof. An effective amount, which may also be referred to as a therapeutically effective amount, refers to the amount (e.g., dose) at which a desired clinical result (e.g., expression of a protein) is achieved in a subject. An effective amount is based, at least in part, on the target tissue, target cell type, means of administration, physical characteristics of the inhibitor, other components of the composition, and other determinants, such as age, body weight, height, sex, and general health of the subject.

Some aspects of the disclosure relate to a method of treating a disease or condition in the subject. In some embodiments, the subject has or is at risk of having a disease or condition.

A subject may be a mammal, such as a human, a non-human primate (e.g., Rhesus monkey, chimpanzee), or a rodent (e.g., a mouse or a rat). In some embodiments, the subject is a human subject.

In some embodiments of the methods provided herein, the vector or markerless DNA is administered via injection or infusion. Administration by injection is a process in which a vector or markerless DNA is delivered to a subject through an apparatus. Injection may deliver a composition to a muscle (intramuscular), into the peritoneal cavity (intraperitoneal), or under the skin (subcutaneous) Administration by infusion is a process in which a vector or markerless DNA is delivered to a subject in a controlled manner over a period of time, such as by a needle or catheter. Infusion may deliver a composition directly to the bloodstream (intravenous) or under the skin (subcutaneous).

### Lipid Compositions

In some embodiments, the nucleic acids are formulated as a lipid composition, such as a composition comprising a lipid nanoparticle, a liposome, and/or a lipoplex. In some embodiments, nucleic acids are formulated as lipid nanoparticle (LNP) compositions. Lipid nanoparticles typically comprise amino lipid, non-cationic lipid, structural lipid, and PEG lipid components along with the nucleic acid cargo of interest. The lipid nanoparticles can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2017/038426; PCT/US2014/027077; PCT/US2014/055394; PCT/US2016/052117; PCT/US2012/069610; PCT/US2017/027492; PCT/US2016/059575; PCT/US2016/069491; PCT/US2016/069493; and PCT/US2014/066242, all of which are incorporated by reference herein in their entirety.

In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid, 5-25% non-cationic lipid, 25-55% structural lipid, and 0.5-15% PEG-modified lipid.

In some embodiments, the lipid nanoparticle comprises a molar ratio of 20-60% ionizable amino lipid, 5-30% non-cationic lipid, 10-55% structural lipid, and 0.5-15% PEG-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

### Ionizable Amino Lipids

In some embodiments, the ionizable amino lipid is a compound of Formula (AI):

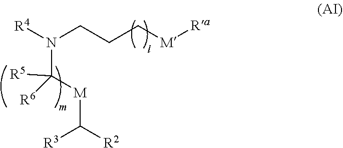

or its N-oxide, or a salt or isomer thereof, wherein R′^{a} is R′^{branched}; wherein
R′^{branched} is:

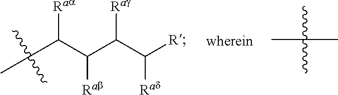

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

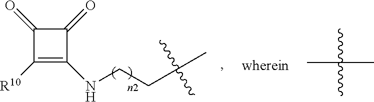

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (AI), R′^{a} is R′^{branched}; R′^{branched} is

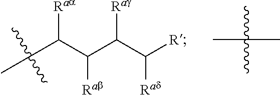

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}; R′^{branched} is

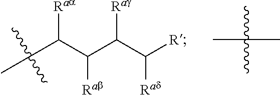

W denotes a point of attachment; R^{aα}, R^{aβ}, R^{aγ}, and R^{aδ} are each H; R and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (AI), R′^{a} is R′^{branched}; R′^{branched} is

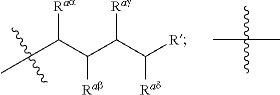

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

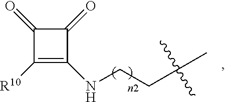

R¹⁰NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (I), R′^{a} is R′^{branched}; R′^{branched} is

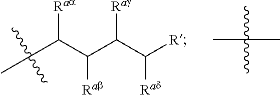

denotes a point of attachment; R^{aα}, R^{aβ}, and R^{aδ} are each H; Ray is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the compound of Formula (I) is selected from:

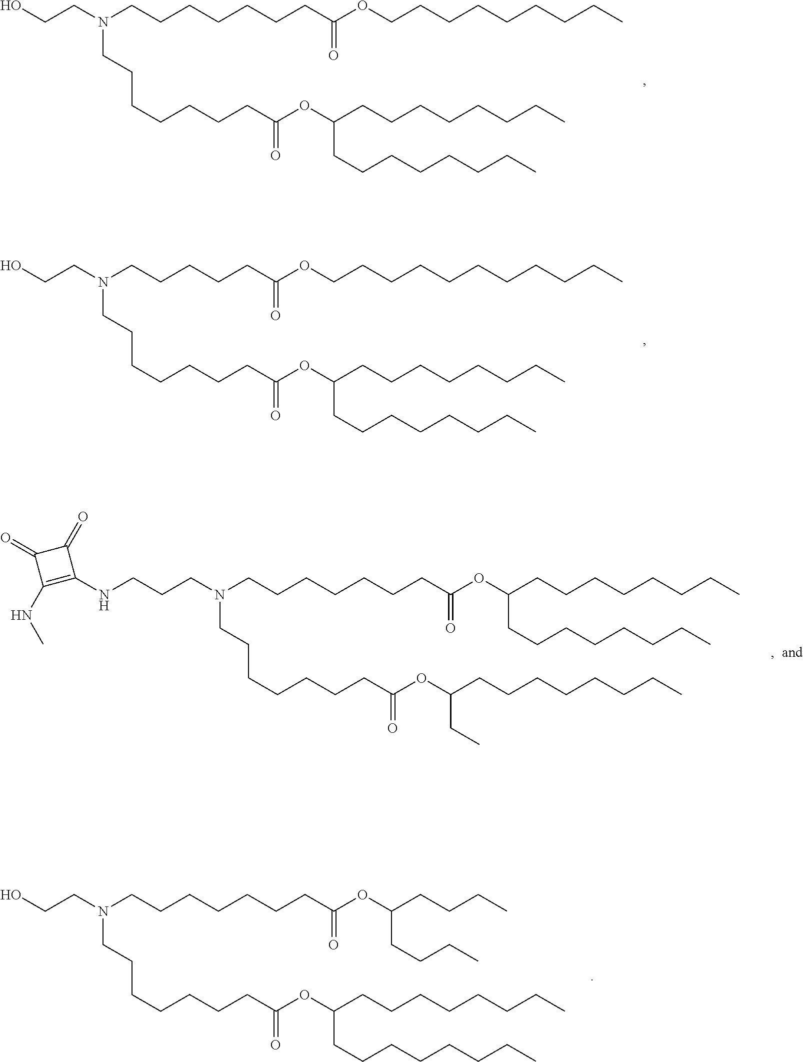 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OC(CCCC)CCCC]

In some embodiments, the ionizable amino lipid is a compound of Formula (AIa):

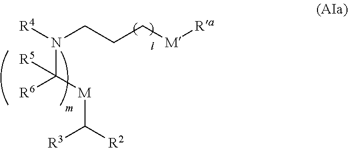

or its N-oxide, or a salt or isomer thereof,
wherein R′^{a} is R′^{branched}; wherein
R′^{branched} is:

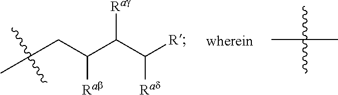

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

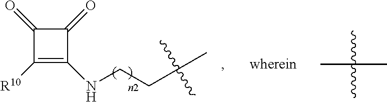

denotes a point of attachment; wherein
   R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid is a compound of Formula (AIb):

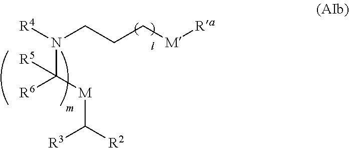

or its N-oxide, or a salt or isomer thereof, wherein R′^{a} is R′^{branched}, wherein
R′^{branched} is

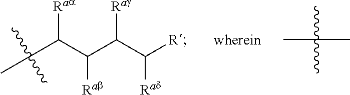

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (AI) or (AIb), R′^{a} is R′^{branched}; R′^{branched} is

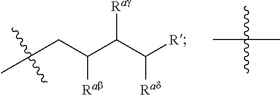

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of Formula (AI) or (AIb), R′^{a} is R′^{branched}; R′^{branched} is

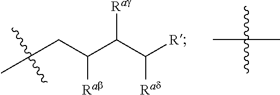

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of Formula (AI) or (AIb), R′^{a} is R′^{branched}; R′^{branched} is

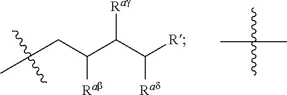

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the ionizable amino lipid is a compound of Formula (AIc):

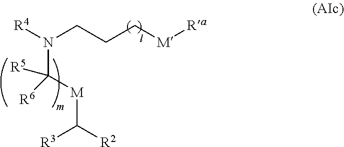

or its N-oxide, or a salt or isomer thereof,
wherein R′^{a} is R′^{branched}; wherein
   R′^{branched} is:

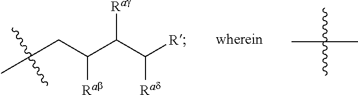

wherein denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

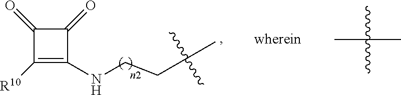

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

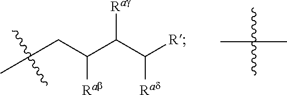

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

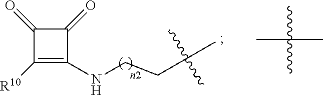

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; 1 is 5; and m is 7.

In some embodiments, the compound of Formula (AIc) is:

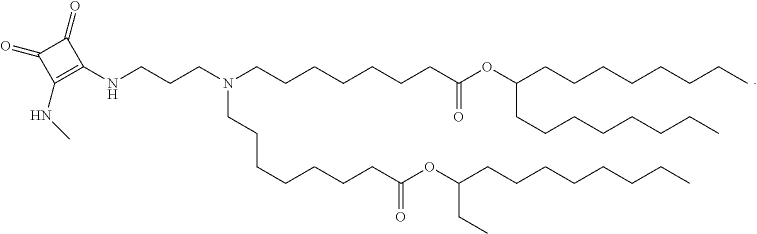 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the ionizable amino lipid is a compound of Formula (AII):

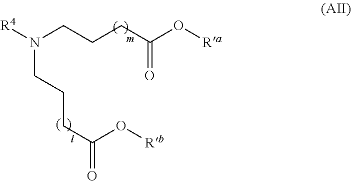

or its N-oxide, or a salt or isomer thereof,
wherein R′^{a} is R′^{branched} or R′^{cyclic}; wherein
R′^{branched} is:

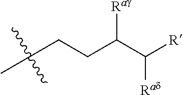

and R′^{cyclic} is:

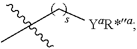

and
R′^{b} is:

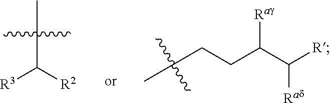

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

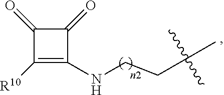

wherein

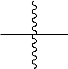

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-a):

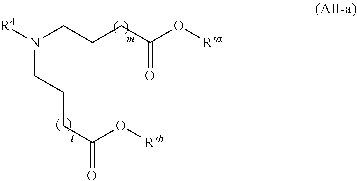

or its N-oxide, or a salt or isomer thereof,
wherein R′^{a} is R′^{branched} or R′^{cyclic}; wherein
R′^{branched} is

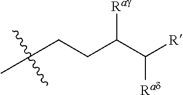

and R′^{b} is:

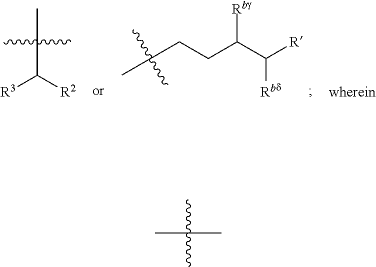

wherein denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

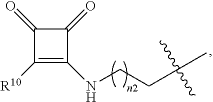

wherein

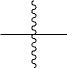

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-b):

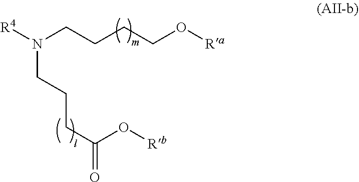

or its N-oxide, or a salt or isomer thereof,
wherein R′^{a} is R′^{branched} or R′^{cyclic}; wherein
R′^{branched} is:

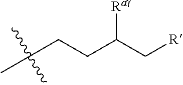

and R′^{b} is:

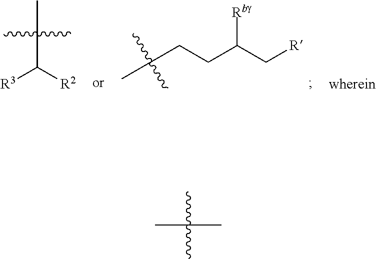

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

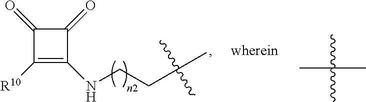

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-c):

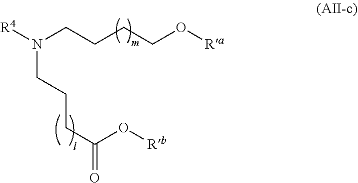

or its N-oxide, or a salt or isomer thereof,
wherein R′^{a} is R′^{branched} or R′^{cyclic}; wherein
R′^{branched} is:

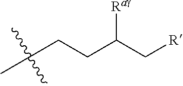

and R′^{b} is:

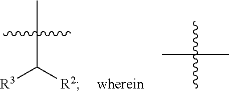

wherein denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

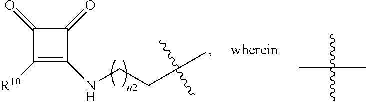

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-d):

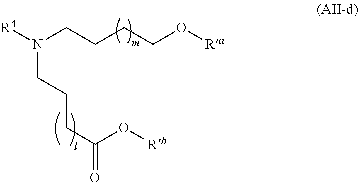

or its N-oxide, or a salt or isomer thereof,
wherein R′^{a} is R′^{branched} or R′^{cyclic}; wherein
R′^{branched} is:

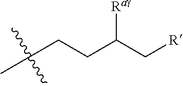

and R′^{b} is:

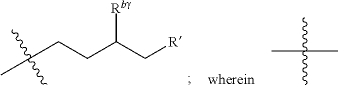

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

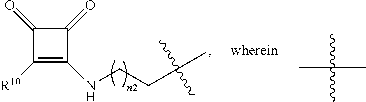

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-e):

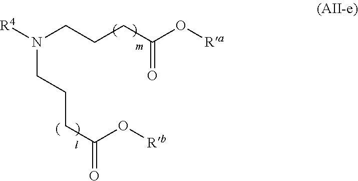

or its N-oxide, or a salt or isomer thereof, wherein R′^{a} is R′^{branched} or R′^{cyclic}; wherein
R′^{branched} is:

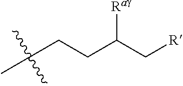

and R′^{b} is:

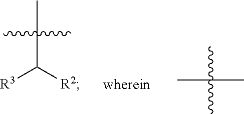

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (AII), (AII-a), (AII-b), (AII-c), (AII-d), or (AII-e), m and l are each independently selected from 4, 5, and 6. In some embodiments of the compound of Formula (AII), (AII-a), (AII-b), (AII-c), (AII-d), or (AII-e), m and l are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C₂₋₅ alkyl.

In some embodiments of the compound of Formula (AII), (AII-a), (AII-b), (AII-c), (AII-d), or (AII-e), R′^{b} is:

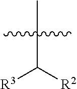

and R² and R³ are each independently a C₁₋₁₄ alkyl. In some embodiments of the compound of Formula (AII), (AII-a), (AII-b), (AII-c), (AII-d), or (AII-e), R′^{b} is:

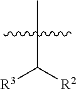

and R² and R³ are each independently a C₆₋₁₀ alkyl. In some embodiments of the compound of Formula (AII), (AII-a), (AII-b), (AII-c), (AII-d), or (AII-e), R′^{b} is:

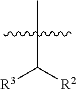

and R² and R³ are each a C₈ alkyl.

In some embodiments of the compound of Formula (AII), (AII-a), (AII-b), (AII-c), (AII-d), or (AII-e), R′^{branched} is:

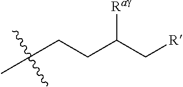

and R′^{b} is:

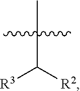

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

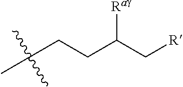

and R′^{b} is:

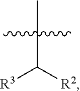

R^{aγ} is a C₂₋₆ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

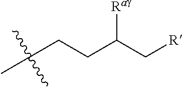

and R′^{b} is:

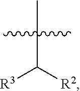

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

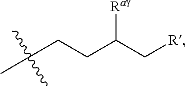

R′^{b} is:

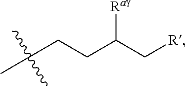

and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

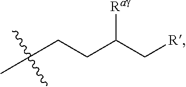

R′^{b} is:

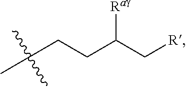

and R^{aγ} and R^{bγ} are each a C₂₋₆ alkyl.

In some embodiments of the compound of Formula (AII), (AII-a), (AII-b), (AII-c), (AII-d), or (AII-e), m and l are each independently selected from 4, 5, and 6 and each R′ independently is a C₁₋₁₂ alkyl. In some embodiments of the compound of Formula (AII), (AII-a), (AII-b), (AII-c), (AII-d), or (AII-e), m and l are each 5 and each R′ independently is a C₂₋₅ alkyl.

In some embodiments of the compound of (AII), (AII-a), (AII-b), (AII-c), (AII-d), or (AII-e), R′^{branched} is:

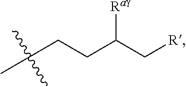

R′^{b} is:

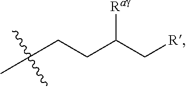

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

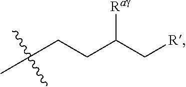

R′^{b} is:

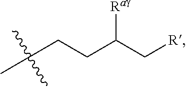

m and l are each 5, each R′ independently is a C₂₋₅ alkyl, and R^{aγ} and R^{bγ} are each a C₂₋₆ alkyl.

In some embodiments of the compound of Formula (AII), (AII-a), (AII-b), (AII-c), (AII-d), or (AII-e), R′^{branched} is:

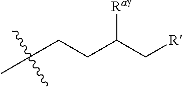

and R′^{b} is:

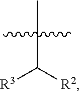

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (AII), (AII-a), (AII-b), (AII-c), (AII-d) or (AII-e), R′^{breached} is:

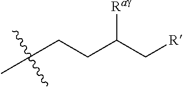

and R′^{b} is:

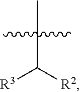

m and l are each 5, R′ is a C₂₋₅ alkyl, R^{aγ} is a C₂₋₆ alkyl, and R² and R³ are each a C₈ alkyl.

In some embodiments of the compound of (AII), (AII-a), (AII-b), (AII-c), (AII-d), or (AII-e), R⁴ is

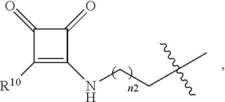

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

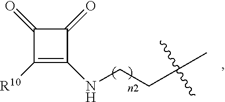

wherein R¹⁰ is NH(CH₃) and n2 is 2.

In some embodiments of the compound of Formula (AII), (AII-a), (AII-b), (AII-c), (AII-d), or (AII-e), R′^{branched} is:

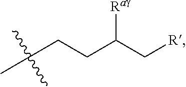

R′^{b} is:

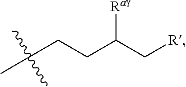

m and l are each independently selected from 4, 5, and 6, each R′ independently is a C₁₋₁₂ alkyl, R^{aγ} and R^{bγ} are each a C₁₋₁₂ alkyl, and R⁴ is

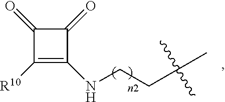

wherein R¹⁰ is NH(C₁₋₆ alkyl), and n2 is 2. In some embodiments of the compound of Formula (AII), (AII-a), (AII-b), (AII-c), (AII-d), or (AII-e), R′^{branched} is:

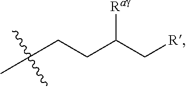

R′^{b} is:

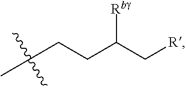

m and l are each 5, each R′ independently is a C₂₋₅ alkyl, R^{aγ} and R^{bγ} are each a C₂₋₆ alkyl, and R⁴ is

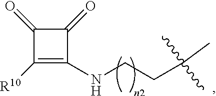

wherein R¹⁰ is NH(CH₃) and n2 is 2.

In some embodiments of the compound of Formula (AII), (AII-a), (AII-b), (AII-c), (AII-d), or (AII-e), R′^{branched} is:

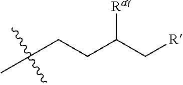

and R′^{b} is:

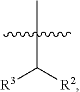

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

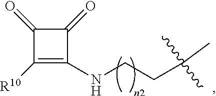

, wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is:

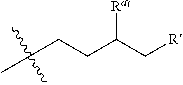

and R′^{b} is:

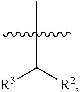

m and l are each 5, R′ is a C₂₋₅ alkyl, R^{aγ} is a C₂₋₆ alkyl, R² and R³ are each a C₈ alkyl, and R⁴ is

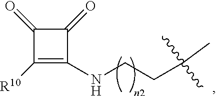

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂)ₙOH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

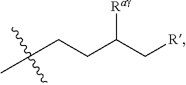

R′^{b} is:

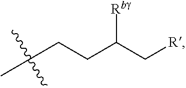

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AII), (AII-a), (AII-b), (AII-c), (AII-d), or (AII-e), R′^{branched} is:

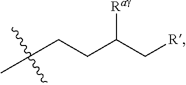

R′^{b} is:

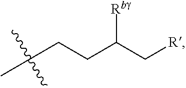

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid is a compound of Formula (AII-f):

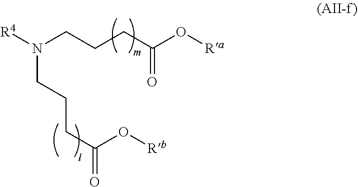

or its N-oxide, or a salt or isomer thereof,
wherein R′^{a} is R′^{branched} or R′^{cyclic}; wherein
R′^{breached} is:

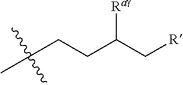

and R′^{b} is:

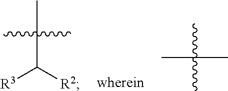

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (AII-f), m and l are each 5, and n is 2, 3, or 4.

In some embodiments of the compound of Formula (AII-f) R′ is a C₂₋₅ alkyl, R^{aγ} is a C₂₋₆ alkyl, and R² and R³ are each a C₆₋₁₀ alkyl.

In some embodiments of the compound of Formula (AII-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the ionizable amino lipid is a compound of Formula (AII-g):

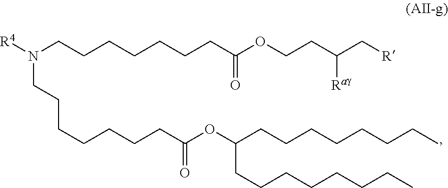

wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

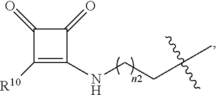

wherein

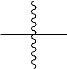

denotes a point of attachment, R¹⁰ is NH(C₁₋₆ alkyl), and n2 is selected from the group consisting of 1, 2, and 3.

In some embodiments, the ionizable amino lipid is a compound of Formula (AII-h):

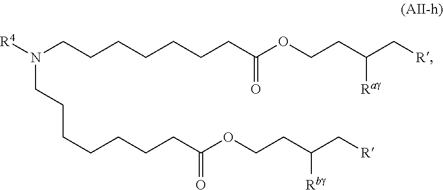

wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

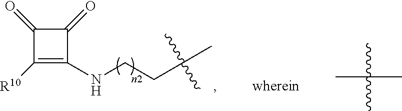

denotes a point of attachment, R¹⁰ is NH(C₁₋₆ alkyl), and n2 is selected from the group consisting of 1, 2, and 3.

In some embodiments of the compound of Formula (AII-g) or (AII-h), R⁴ is

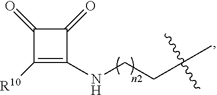

wherein
R¹⁰ is NH(CH₃) and n2 is 2.

In some embodiments of the compound of Formula (AII-g) or (AII-h), R⁴ is —(CH₂)₂OH.

In some embodiments, the ionizable amino lipids may be one or more of compounds of Formula (VI):

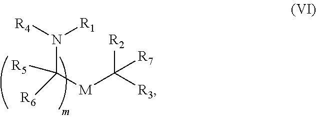

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙ N(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R⁸, —N(R)S(O)₂R⁸, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″—C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′ R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′ R′;
R₂ and R₃ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′ R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R⁴ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R⁴ is selected from the group consisting of —(CH₂)ₙQ, —(CH₂)·CHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R⁷ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In certain embodiments, a subset of compounds of Formula (VI) includes those of Formula (VI-A):

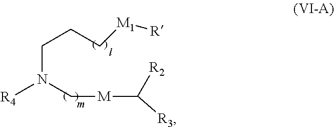

or its N-oxide, or a salt or isomer thereof, wherein l is selected from 1, 2, 3, 4, and 5; m is selected from 5, 6, 7, 8, and 9; M₁ is a bond or M′; R⁴ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″—C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VI-B):

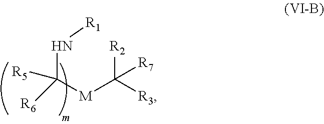

or its N-oxide, or a salt or isomer thereof in which all variables are as defined herein. For example, m is selected from 5, 6, 7, 8, and 9; R⁴ is hydrogen, unsubstituted C₁₋₃ alkyl, or —(CH₂)ₙQ, in which Q is H, —NHC(S)N(R)₂, —NHC(O)N(R)₂, —N(R)C(O)R, —N(R)S(O)₂R, —N(R)R₈, —NHC(═NR₉)N(R)₂, —NHC(=CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″—C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VII):

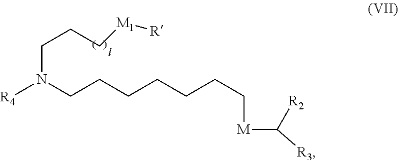

or its N-oxide, or a salt or isomer thereof, wherein l is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(=NR₉)N(R)₂, —NHC(=CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″—C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, the compounds of Formula (VI) are of Formula (VIIa),

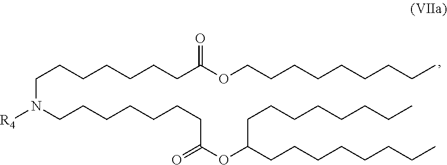

or their N-oxides, or salts or isomers thereof, wherein R₄ is as described herein.

In another embodiment, the compounds of Formula (VI) are of Formula (VIIb),

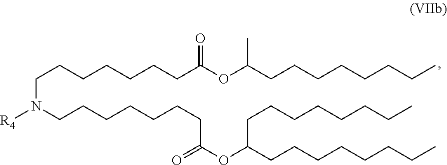

or their N-oxides, or salts or isomers thereof, wherein R₄ is as described herein.

In another embodiment, the compounds of Formula (VI) are of Formula (VIIc) or (VIIe):

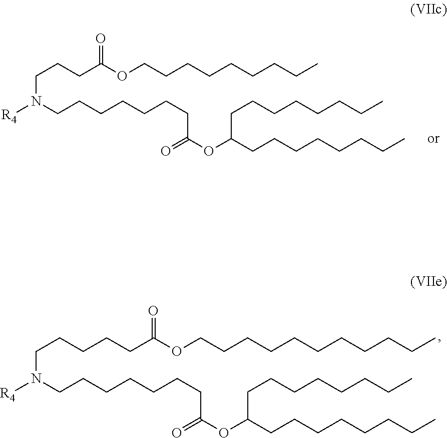

or their N-oxides, or salts or isomers thereof, wherein R₄ is as described herein.

In another embodiment, the compounds of Formula (VI) are of Formula (VIIf):

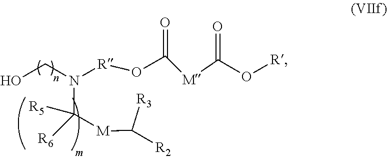

or their N-oxides, or salts or isomers thereof,
wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (VI) are of Formula (VIId),

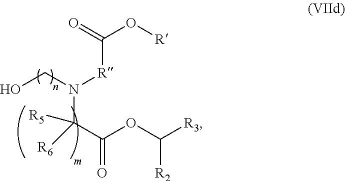

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

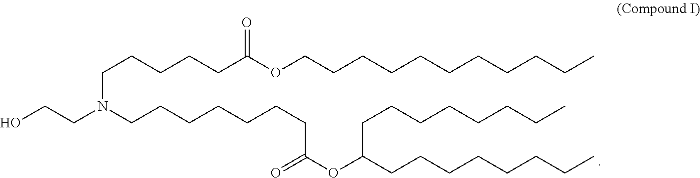 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

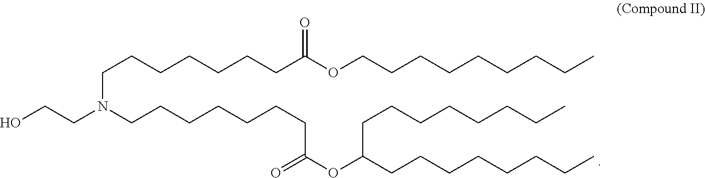 [SMILES: CCCCCCCCCOC(=O)CCCCCCCN(CCO)CCCCCCCC(=O)OC(CCCCCCCC)CCCCCCCC]

In a further embodiment, the compounds of Formula (VI) are of Formula (VIIg),

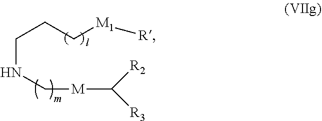

or their N-oxides, or salts or isomers thereof, wherein l is selected from 1, 2, 3, 4, and 5; m is selected from 5, 6, 7, 8, and 9; M₁ is a bond or M′; M and M′ are independently selected from —C(O)O—, —OC(O)—, —OC(O)-M″—C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

The central amine moiety of a lipid according to Formula (VI), (VI-A), (VI-B), (VII), (VIIa), (VIIb), (VIIc), (VIId), (VIIe), (VIIf), or (VIIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some embodiments, the ionizable amino lipids may be one or more of compounds of formula (VIII),

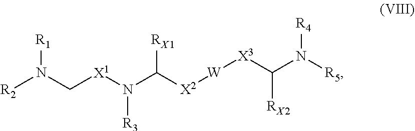

or salts or isomers thereof, wherein
W is

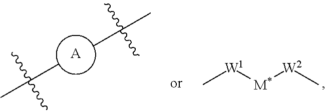

ring A is

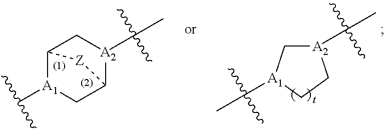

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R⁶)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

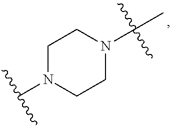 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (VIIIa1)-(VIIIa8):

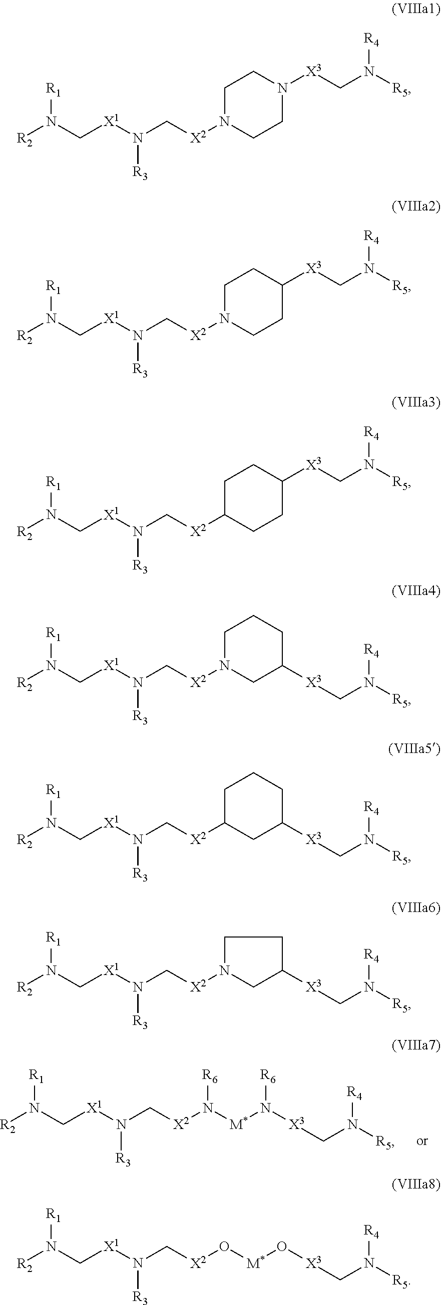

In some embodiments, the ionizable amino lipid is

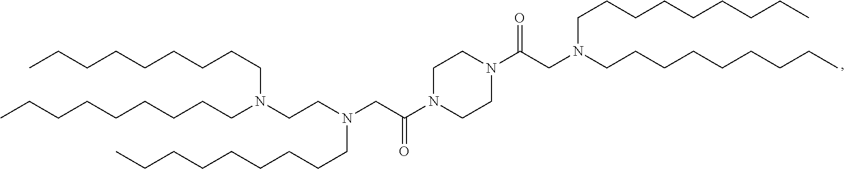 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (VIII), (VIIIa1), (VIIIa2), (VIIIa3), (VIIIa4), (VIIIa5), (VIIIa6), (VIIIa7), or (VIIIa8) may be protonated at a physiological pH. Thus, a lipid may have a positive or partial positive charge at physiological pH.

In some embodiments, the lipid nanoparticle comprises a lipid having the structure:

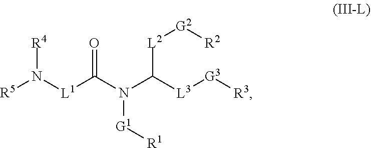

or a pharmaceutically acceptable salt, tautomer, or stereoisomer thereof, wherein:
   R¹ is optionally substituted C₁-C₂₄ alkyl or optionally substituted C₂-C₂₄ alkenyl;
   R² and R³ are each independently optionally substituted C₁-C₃₆ alkyl;
   R⁴ and R⁵ are each independently optionally substituted C₁-C₆ alkyl, or R⁴ and R⁵ join, along with the N to which they are attached, to form a heterocyclyl or heteroaryl;
   L¹, L², and L³ are each independently optionally substituted C₁-C₁₈ alkylene;
   G¹ is a direct bond, —(CH₂)ₙO(C═O)—, —(CH₂)ₙ(C═O)O—, or —(C═O)—;
   G² and G³ are each independently —(C═O)O— or —O(C═O)—; and n is an integer greater than 0.

In some embodiments, the lipid nanoparticle comprises a lipid having the structure:

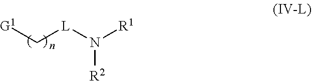

or a pharmaceutically acceptable salt, tautomer, or stereoisomer thereof, wherein:
   G¹ is —N(R³)R⁴ or —OR⁵;
   R¹ is optionally substituted branched, saturated or unsaturated C₁₂-C₃₆ alkyl;
   R² is optionally substituted branched or unbranched, saturated or unsaturated C₁₂-C₃₆alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
   R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
   R⁵ is H or optionally substituted C₁-C₆ alkyl;
   L is —C(═O)—, C₆-C₁₂ alkylene, C₆-C₁₂ alkenylene, or C₂-C₆ alkynylene; and
   n is an integer from 1 to 12.

In some embodiments, the lipid nanoparticle comprises a lipid having the structure:

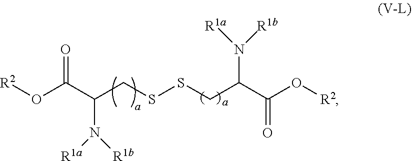

or a pharmaceutically acceptable salt thereof, wherein:
   each R^{1a} is independently hydrogen, R^{1c}, or R^{1d};
   each R^{1b} is independently R^{1c} or R^{1d};
   each R^{1c} is independently —[CH₂]₂C(O)X¹R³;
   each R^{1d} is independently —C(O)R⁴;
   each R² is independently —[C(R^{2a})₂]_{c}R^{2b};
   each R^{2a} is independently hydrogen or C₁-C₆ alkyl;
   R^{b} is —N(L₁-B)₂; —(OCH₂CH₂)₆OH; or —(OCH₂CH₂)_{b}OCH₃;
   each R³ and R⁴ is independently C₆-C₃₀ aliphatic;
   each L₃ is independently C₁-C₁₀ alkylene;
   each B is independently hydrogen or an ionizable nitrogen-containing group;
   each X¹ is independently a covalent bond or 0;
   each a is independently an integer of 1-10;
   each b is independently an integer of 1-10; and
   each c is independently an integer of 1-10.

In some embodiments, the lipid nanoparticle comprises a lipid having the structure:

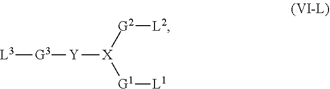

or a pharmaceutically acceptable salt, prodrug or stereoisomer thereof, wherein:
   X is N, and Y is absent; or X is CR, and Y is NR;
   L¹ is —O(C—O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, or —NR^{a}C(═O)OR¹;
   L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
   L³ is —O(C═O)R³ or —(C═O)OR³;
   G¹ and G² are each independently C₂-C₁₂ alkylene or C₂-C₁₂ alkenylene;
   G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
   R^{a}, R^{b}, R^{d} and R^{e} are each independently H or C₁-C₁₂ alkyl or C₁-C₁₂ alkenyl; R′ and R^{f} are each independently C₁-C₁₂ alkyl or C₂-C₁₂ alkenyl;
   each R is independently H or C₁-C₁₂ alkyl;
   R¹, R² and R³ are each independently C₁-C₂₄ alkyl or C₂-C₂₄ alkenyl; and x is 0, 1 or 2, and wherein each alkyl, alkenyl, alkylene, alkenylene, heteroalkylene and heteroalkenylene is independently substituted or unsubstituted unless otherwise specified.

In some embodiments, the lipid nanoparticle comprises a lipid having the structure:

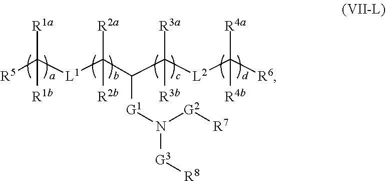

or a pharmaceutically acceptable salt, tautomer, prodrug or stereoisomer thereof, wherein:
   L¹ and L² are each independently —O(C═O)—, —(C═O)O—, —C(═O)—, —O—, —S(O)ₓ—S, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
   G¹ is C₁-C₂ alkylene, —(C═O)—, —O(C═O)—, —SC(═O)—, —NR^{a}C(═O)— or a direct bond;
   G² is —C(O)—, —(CO)O—, —C(═O)S—, —C(═O)NR^{a}— or a direct bond; G³ is C₁-C₆ alkylene;
   R^{a} is H or C₁-C₁₂ alkyl;
   R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
   R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
   R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
   R^{4A} and R^{4B} are, at each occurrence, independently either: (a) H or C₁-C₁₂ alkyl; or (b) R^{4A} is H or C₁-C₁₂ alkyl, and R^{4B} together with the carbon atom to which it is bound is taken together with an adjacent R^{4B} and the carbon atom to which it is bound to form a carbon-carbon double bond;
   R⁵ and R⁶ are each independently H or methyl;
   R⁷ is H or C₁-C₂₀ alkyl;
   R⁸ is OH, —N(R⁹)(C═O)R¹⁰, —(C═O)NR⁹R¹⁰, —NR⁹R¹⁰, —(C═O)OR″ ¹ or —O(C═O)R″, provided that G³ is C₄-C₆ alkylene when R⁸ is —NR⁹R¹⁰,
   R⁹ and R¹⁰ are each independently H or C₁-C₁₂ alkyl;
   R″ is aralkyl;
   a, b, c and d are each independently an integer from 1 to 24; and x is 0, 1 or 2,
   wherein each alkyl, alkylene and aralkyl is optionally substituted.

In some embodiments, the lipid nanoparticle comprises a lipid having the structure:

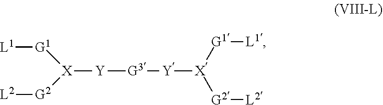

or a pharmaceutically acceptable salt, prodrug or stereoisomer thereof, wherein:
   X and X′ are each independently N or CR;
   Y and Y are each independently absent, —O(C═O)—, —(C═O)O— or NR, provided that:
      a) Y is absent when X is N;
      b) Y′ is absent when X′ is N;
      c) Y is —O(C═O)—, —(C═O)O— or NR when X is CR; and
      d) Y′ is —O(C═O)—, —(C═O)O— or NR when X′ is CR,
   L¹ and L^{1′} are each independently —O(C═O)R′, —(C═O)OR′, —C(═O)R′, —OR¹, —S(O)_{z}R′, —S—SR¹, —C(═O)SR′, —SC(═O)R′, —NR^{a}C(═O)R′, —C(═O)NR^{b}R^{c}, —NR^{a}C(═O)NR^{b}R^{c}, —OC(═O)NR^{b}R^{c} or —NR^{a}C(═O)OR′;
   L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²
   G¹. G^{1′}, G² and G^{2′} are each independently C₂-C₁₂ alkylene or C₂-C₁₂ alkenylene;
   G is C₂-C₂₄ heteroalkylene or C₂-C₂₄ heteroalkenylene;
   R^{a}, R^{b}, R^{d} and R^{e} are, at each occurrence, independently H, C₁-C₁₂ alkyl or C₂-C₁₂ alkenyl;
   R^{c} and R^{f} are, at each occurrence, independently C₁-C₁₂ alkyl or C₂-C₁₂ alkenyl;
   R is, at each occurrence, independently H or C₁-C₁₂ alkyl;
   R¹ and R² are, at each occurrence, independently branched C₆-C₂₄ alkyl or branched C₆-C₂₄ alkenyl;
   z is 0, 1 or 2, and wherein each alkyl, alkenyl, alkylene, alkenylene, heteroalkylene and heteroalkenylene is independently substituted or unsubstituted unless otherwise specified.

In some embodiments, the lipid nanoparticle comprises a lipid having the structure:

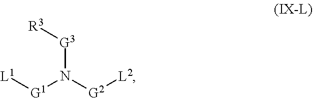

or a pharmaceutically acceptable salt, prodrug or stereoisomer thereof, wherein:
   L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
   L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{c}R^{f}, —OC(═O)NR^{e}R^{f}; —NR^{d}C(═O)OR² or a direct bond to R²;
   G¹ and G² are each independently C₂-C₁₂ alkylene or C₂-C₁₂ alkenylene;
   G³ is C₁-C₂₄ alkylene, C₂-C₂₄ alkenylene, C₃-C₈ cycloalkylene or C₃-C₈ cycloalkenylene;
   R^{a}, R^{b}, R^{d} and R^{e} are each independently H or C₁-C₁₂ alkyl or C₁-C₁₂ alkenyl;
   R^{e} and R^{f} are each independently C₁-C₁₂ alkyl or C₂-C₁₂ alkenyl;
   R¹ and R² are each independently branched C₆-C₂₄ alkyl or branched C₆-C₂₄ alkenyl;
   R³ is —N(R⁴)R⁵;
   R⁴ is C₁-C₁₂ alkyl;
   R⁵ is substituted C₁-C₁₂ alkyl; and
   x is 0, 1 or 2, and
   wherein each alkyl, alkenyl, alkylene, alkenylene, cycloalkylene, cycloalkenylene, aryl and aralkyl is independently substituted or unsubstituted unless otherwise specified.

In some embodiments, the lipid nanoparticle comprises a lipid having the structure:

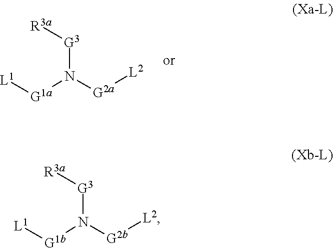

or a pharmaceutically acceptable salt, prodrug or stereoisomer thereof, wherein:
   L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
   L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R².
   G^{1a} and G^{2b} are each independently C₂-C₁₂ alkylene or C₂-C₁₂ alkenylene;
   G^{1b} and G^{2b} are each independently C₁-C₁₂ alkylene or C₂-C₁₂ alkenylene;
   G³ is C₁-C₂₄ alkylene, C₂-C₂₄ alkenylene, C₃-C₈ cycloalkylene or C₃-C₈ cycloalkenylene;
   R^{a}, R^{b}, R^{d} and R^{e} are each independently H or C₁-C₁₂ alkyl or C₂-C₁₂ alkenyl;
   R^{c} and R^{f} are each independently C₁-C₁₂ alkyl or C₂-C₁₂ alkenyl;
   R¹ and R² are each independently branched C₆-C₂₄ alkyl or branched C₆-C₂₄ alkenyl;
   R^{3a} is —C(═O)N(R^{4a})R^{5a} or —C(═O)OR⁶;
   R^{3b} is —NR^{4b}C(═O)R^{5b};
   R^{4a} is C₁-C₁₂ alkyl;
   R^{4b} is H, C₁-C₁₂ alkyl or C₂-C₁₂ alkenyl;
   R^{5a} is H, C₁-C₈ alkyl or C₂-C₈ alkenyl;
   R^{5b} is C₂-C₁₂ alkyl or C₂-C₁₂ alkenyl when R^{4b} is H; or R^{5b} is C₁-C₁₂ alkyl or C₂-C₁₂ alkenyl when R^{4b} is C₁-C₁₂ alkyl or C₂-C₁₂ alkenyl;
   R⁶ is H, aryl or aralkyl; and
   x is 0, 1 or 2, and
   wherein each alkyl, alkenyl, alkylene, alkenylene, cycloalkylene, cycloalkenylene, aryl and aralkyl is independently substituted or unsubstituted.

In some embodiments, the lipid nanoparticle comprises a lipid having the structure:

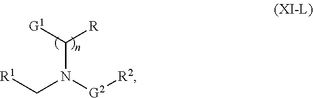

or a pharmaceutically acceptable salt, prodrug or stereoisomer thereof, wherein:
   G¹ is —OH, —R³R⁴, —(C═O)R⁵ or —R³(C═O)R⁵;
   G² is —CH₂— or —(C═O)—;
   R is, at each occurrence, independently H or OH;
   R¹ and R² are each independently optionally substituted branched, saturated or unsaturated C₁₂-C₃₆ alkyl;
   R³ and R⁴ are each independently H or optionally substituted straight or branched, saturated or unsaturated C₁-C₆ alkyl;
   R⁵ is optionally substituted straight or branched, saturated or unsaturated C₁-C₆ alkyl; and
   n is an integer from 2 to 6.

In some embodiments, the lipid nanoparticle comprises a lipid having the structure:

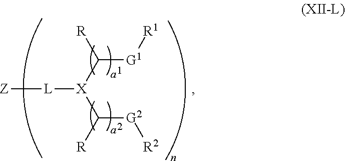

or a pharmaceutically acceptable salt, prodrug or stereoisomer thereof, wherein:
   one of G¹ or G² is, at each occurrence, —O(C═O)—, —(C═O)O—, —C(═O)—, —O—, —S(O), —S—S—, —C(═O)S—, SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;
   L is, at each occurrence, —O(C═O)—, wherein ˜ represents a covalent bond to X; X is CR^{a};
   Z is alkyl, cycloalkyl or a monovalent moiety comprising at least one polar functional group when n is 1; or Z is alkylene, cycloalkylene or a polyvalent moiety comprising at least one polar functional group when n is greater than 1;
   R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
   R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
   R¹ and R² have, at each occurrence, the following structure, respectively:

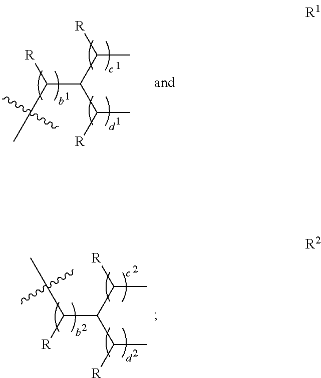

a¹ and a² are, at each occurrence, independently an integer from 3 to 12; b¹ and b² are, at each occurrence, independently 0 or 1;
c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In some embodiments, the lipid nanoparticle comprises a lipid having the structure:

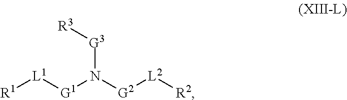

or a pharmaceutically acceptable salt, prodrug or stereoisomer thereof, wherein:
   one of L¹ or L² is —O(C═O)—, —(C═O)O—, —C(═O)—, —O—, —S(O)X—, —S—S—, —C(═O)S—, —SC(═O)—, —R^{a}C(═O)—, —C(═O)R^{a}—, R^{a}C(═O)R^{a}—, —OC(═O)R^{a}— or —R^{a}C(═O)O—, and the other of L¹ or L² is —O(C═O)—, —(C═O)O—, —C(═O)—, —O—, —S(O)ₓ—, —S—S—, —C(═O)S—, SC(═O)—, —R^{a}C(═O)—, —C(═O)R^{a}—, R^{a}C(═O)R^{a}—, —OC(═O)R^{a}— or —NR^{a}C(═O)O— or a direct bond;
   G¹ and G² are each independently unsubstituted C₁-C₁₂ alkylene or C₁-C₁₂ alkenylene;
   G³ is C₁-C₂₄ alkylene, C₁-C₂₄ alkenylene, C₃-C₈ cycloalkylene, C₃-C₈ cycloalkenylene;
   R^{a} is H or C₁-C₁₂ alkyl;
   R¹ and R² are each independently C₆-C₂₄ alkyl or C₆-C₂₄ alkenyl;
   R³ is H, OR⁵, CN, —C(═O)OR⁴, —OC(═O)R⁴ or —R⁵C(═O)R⁴;
   R⁴ is C₁-C₁₂ alkyl;
   R⁵ is H or C₁-C₆ alkyl; and
   x is 0, 1 or 2.

In some embodiments, the lipid nanoparticle comprises a lipid having the structure:

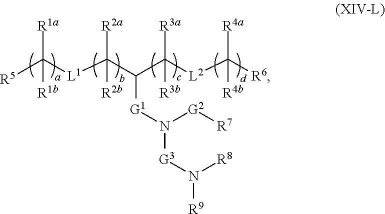

or a pharmaceutically acceptable salt, tautomer, prodrug or stereoisomer thereof, wherein:
   L¹ and L² are each independently —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, —R^{a}C(═O)R^{a}—, —OC(═O)R^{a}—, —R^{a}C(═O)O— or a direct bond;
   G¹ is C₁-C₂ alkylene, —(C═O)—, —O(C═O)—, —SC(═O)—, —R^{a}C(═O)— or a direct bond;
   G² is —C(═O)—, —(C═O)O—, —C(═O)S—, —C(═O)NR^{a}— or a direct bond;
   G³ is C₁-C₆ alkylene;
   R^{a} is H or C₁-C₁₂ alkyl;
   R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
   R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
   R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
   R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
   R⁵ and R⁶ are each independently H or methyl;
   R⁷ is C₄-C₂₀ alkyl;
   R⁸ and R⁹ are each independently C₁-C₁₂ alkyl; or R⁸ and R⁹, together with the nitrogen atom to which they are attached, form a 5, 6 or 7-membered heterocyclic ring; a, b, c and d are each independently an integer from 1 to 24; and x is 0, 1 or 2.

In some embodiments, the lipid nanoparticle comprises a lipid having the structure:

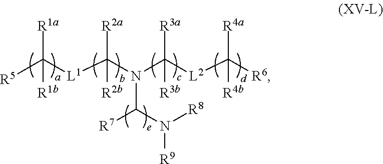

or a pharmaceutically acceptable salt, tautomer, prodrug or stereoisomer thereof, wherein:
   L¹ and L² are each independently —O(C═O)—, —(C═O)O— or a carbon-carbon double bond;
   R^{1a} and R^{1b} are, at each occurrence, independently either (a) H or C₁-C₁₂ alkyl, or (b)R^{1a} is H or C₁-C₁₂ alkyl, and R^{1b} together with the carbon atom to which it is bound is taken together with an adjacent R^{1b} and the carbon atom to which it is bound to form a carbon-carbon double bond;
   R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
   R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
   R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
   R⁵ and R⁶ are each independently methyl or cycloalkyl;
   R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
   a and d are each independently an integer from 0 to 24; b and c are each independently an integer from 1 to 24; and e is 1 or 2,
   provided that:
   at least one of R^{1a}, R^{2a}, R^{3a} or R^{4a} is C₁-C₁₂ alkyl, or at least one of L¹ or L² is —O(C═O)— or —(C═O)O—; and
   R^{1a} and R^{1b} are not isopropyl when a is 6 or n-butyl when a is 8.

In some embodiments, the lipid nanoparticle comprises a lipid having the structure:

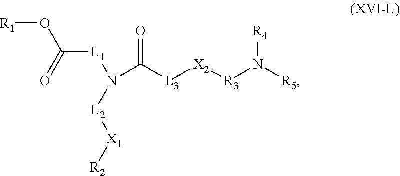

or a pharmaceutically acceptable salt thereof, wherein
   R₁ and R₂ are the same or different, each a linear or branched alkyl with 1-9 carbons, or as alkenyl or alkynyl with 2 to 11 carbon atoms,
   L₁ and L₂ are the same or different, each a linear alkyl having 5 to 18 carbon atoms, or form a heterocycle with N,
   X₁ is a bond, or is —CG-G- whereby L₂-CO—O—R₂ is formed,
   X₂ is S or O,
   L₃ is a bond or a lower alkyl, or form a heterocycle with N,
   R₃ is a lower alkyl, and
   R₄ and R₅ are the same or different, each a lower alkyl.

In some embodiments, the lipid nanoparticle comprises an ionizable lipid having the structure:

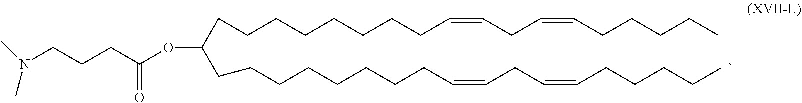 [SMILES: CCCCC/C=C\C/C=C\CCCCCCCCC(CCCCCCCC/C=C\C/C=C\CCCCC)OC(=O)CCCN(C)C]

or a pharmaceutically acceptable salt thereof.

In some embodiments, the lipid nanoparticle comprises a lipid having the structure:

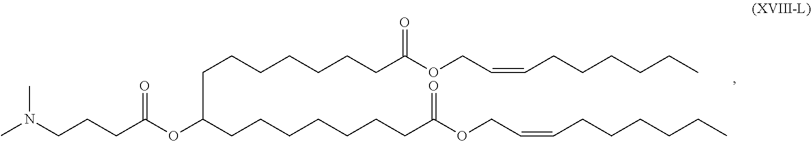 [SMILES: CCCCCC/C=C\COC(=O)CCCCCCCC(CCCCCCCC(=O)OC/C=C\CCCCCC)OC(=O)CCCN(C)C]

or a pharmaceutically acceptable salt thereof.

In some embodiments, the lipid nanoparticle comprises a lipid having the structure:

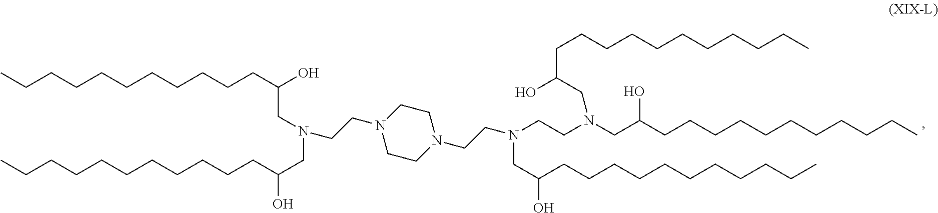 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

or a pharmaceutically acceptable salt thereof.

In some embodiments, the lipid nanoparticle comprises a lipid having the structure:

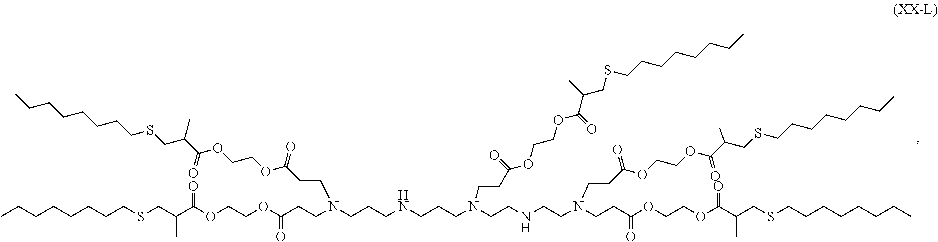 [SMILES: CCCCCCCCSCC(C)C(=O)OCCOC(=O)CCN(CCCNCCCN(CCC(=O)OCCOC(=O)C(C)CSCCCCCCCC)CCC(=O)OCCOC(=O)C(C)CSCCCCCCCC)CCNCCN(CCC(=O)OCCOC(=O)C(C)CSCCCCCCCC)CCC(=O)OCCOC(=O)C(C)CSCCCCCCCC]

or a pharmaceutically acceptable salt thereof.

In some embodiments, the lipid nanoparticle comprises a lipid having the structure:

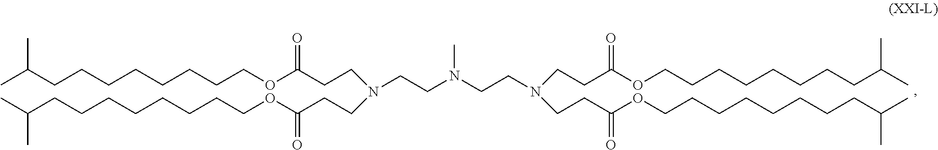 [SMILES: CC(C)CCCCCCCCOC(=O)CCN(CCC(=O)OCCCCCCCCC(C)C)CCN(C)CCN(CCC(=O)OCCCCCCCCC(C)C)CCC(=O)OCCCCCCCCC(C)C]

or a pharmaceutically acceptable salt thereof.

In some embodiments, the lipid nanoparticle comprises a lipid having the structure:

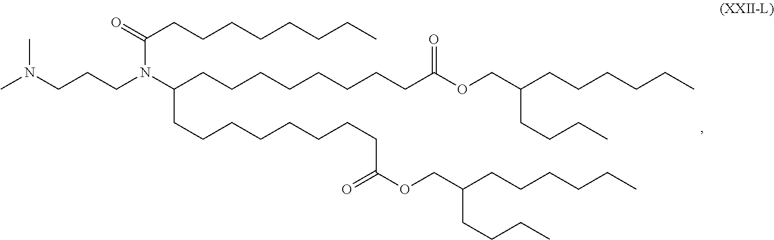 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

or a pharmaceutically acceptable salt thereof.

In some embodiments, the lipid nanoparticle comprises a lipid having the structure:

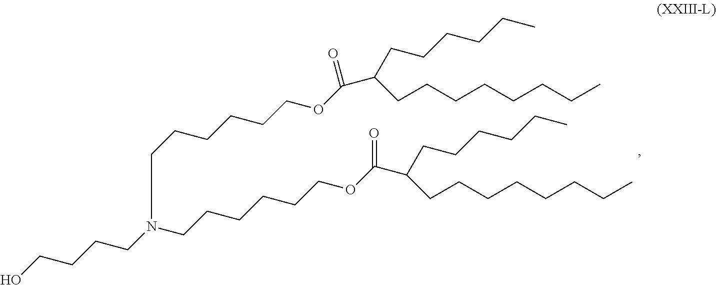 [SMILES: CCCCCCCCC(CCCCCC)C(=O)OCCCCCCN(CCCCO)CCCCCCOC(=O)C(CCCCCC)CCCCCCCC]

or a pharmaceutically acceptable salt thereof.

In some embodiments the lipid nanoparticle comprises a lipid having the structure:

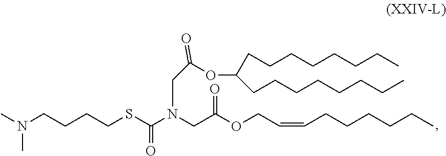 [SMILES: CCCCCC/C=C\COC(=O)CN(CC(=O)OC(CCCCCCCC)CCCCCCCC)C(=O)SCCCCN(C)C]

or a pharmaceutically acceptable salt thereof.

In some embodiments, the lipid nanoparticle comprises a lipid having the structure:

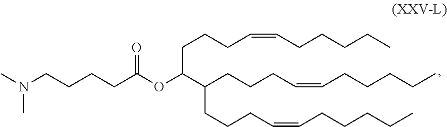 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments, the lipid nanoparticle comprises a lipid having the structure:

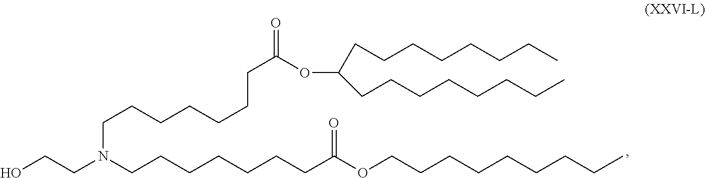 [SMILES: CCCCCCCCCOC(=O)CCCCCCCN(CCO)CCCCCCCC(=O)OC(CCCCCCCC)CCCCCCCC]

or a pharmaceutically acceptable salt thereof.

In some embodiments, the lipid nanoparticle comprises a lipid having the structure:

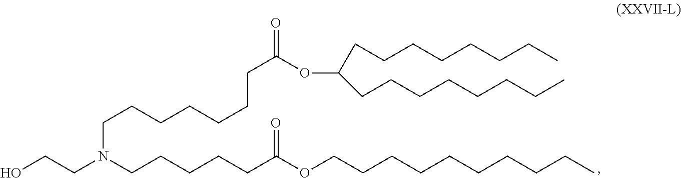 [SMILES: CCCCCCCCCCOC(=O)CCCCCN(CCO)CCCCCCCC(=O)OC(CCCCCCCC)CCCCCCCC]

or a pharmaceutically acceptable salt thereof.

### Non-Cationic Lipids

In certain embodiments, the lipid nanoparticles described herein comprise one or more non-cationic lipids. Non-cationic lipids may be phospholipids.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In certain embodiments, the lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In certain embodiments, a phospholipid is an analog or variant of DSPC. In certain embodiments, a phospholipid is a compound of Formula (IX):

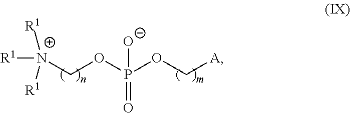

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

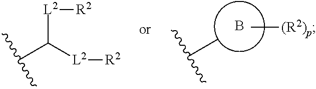

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
   Ring B is optionally substituted carbocyclyl, optionally substituted heterocyclyl, optionally substituted aryl, or optionally substituted heteroaryl; and
p is 1 or 2;
provided that the compound is not of the formula:

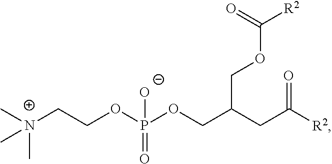

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

In some embodiments, the lipid nanoparticle comprises a molar ratio of 5-25% non-cationic lipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% non-cationic lipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% phospholipid lipid.

### Structural Lipids

The lipid composition of a pharmaceutical composition disclosed herein can comprise one or more structural lipids. As used herein, the term “structural lipid” includes sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises 30-45 mol % sterol, optionally 35-40 mol %, for example, 30-31 mol %, 31-32 mol %, 32-33 mol %, 33-34 mol %, 35-35 mol %, 35-36 mol %, 36-37 mol %, 38-38 mol %, 38-39 mol %, or 39-40 mol %. In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or 40 mol % cholesterol.

### Polyethylene Glycol (PEG)-Lipids

The lipid composition of a pharmaceutical composition disclosed herein can comprise one or more polyethylene glycol (PEG) lipids.

As used herein, the term “PEG-lipid” or “PEG-modified lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG, and/or PEG-DPG.

In some embodiments, the lipid moiety of the PEG-lipids includes those having lengths of from about C₁, to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

In some embodiments, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG. PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

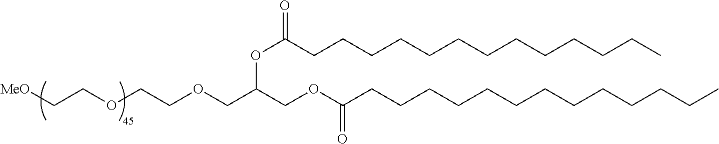

In some embodiments, PEG lipids can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

In certain embodiments, a PEG lipid is a compound of Formula (X):

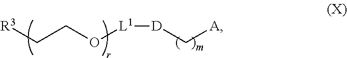

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N}); D is a moiety obtained by click chemistry or a moiety cleavable under physiological conditions;
m is 0, 1, 2, 3, 4, 5, 6, 7, 8, 9, or 10;
A is of the formula:

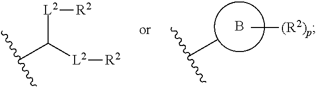

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
   Ring B is optionally substituted carbocyclyl, optionally substituted heterocyclyl, optionally substituted aryl, or optionally substituted heteroaryl; and
   p is 1 or 2.

In certain embodiments, the compound of Formula (X) is a PEG-OH lipid (i.e., R³ is —OR^{O}, and R^{O} is hydrogen). In certain embodiments, the compound of Formula (X) is of Formula (X—OH):

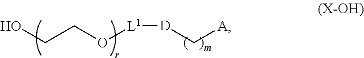

or a salt thereof.

In certain embodiments, a PEG lipid is a PEGylated fatty acid. In certain embodiments, a PEG lipid is a compound of Formula (XI). Provided herein are compounds of Formula (XI):

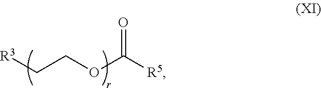

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (XI) is of Formula (XI-OH):

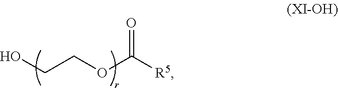

or a salt thereof. In some embodiments, r is 40-50.

In yet other embodiments the compound of Formula (XI) is:

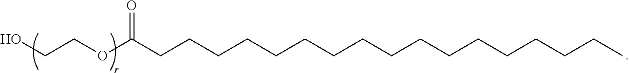

or a salt thereof.

In some embodiments the compound of Formula (XI) is

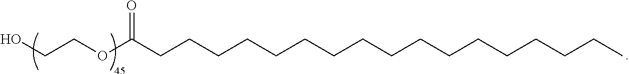

In some embodiments, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises 1-5% PEG-modified lipid, optionally 1-3 mol %, for example 1.5 to 2.5 mol %, 1-2 mol %, 2-3 mol %, 3-4 mol %, or 4-5 mol %. In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

Some embodiments comprise adding PEG to a composition comprising an LNP encapsulating a nucleic acid (e.g., which already includes PEG in the amounts listed above). Without being bound by theory, it is believed that spiking a LNP composition with additional PEG can provide benefits during lyophilization. Thus, some embodiments, comprise adding additional PEG as compared to an amount used for a non-lyophilized LNP composition. In embodiments comprise adding about 0.5 mo % or more PEG to an LNP composition, such as about 1 mol %, about 1.5 mol %, about 2 mol %, about 2.5 mol %, about 3 mol %, about 3.5 mol %, about 4 mol %, about 5 mol %, or more after formation of an LNP composition (e.g., which already contains PEG in amount listed elsewhere herein).

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, a LNP comprises an ionizable amino lipid of Compound 1, wherein the non-cationic lipid is DSPC, the structural lipid that is cholesterol, and the PEG lipid is DMG-PEG.

In some embodiments, a LNP comprises an ionizable amino lipid of any of Formula VI, VII or VIIII, a phospholipid comprising DSPC, a structural lipid, and a PEG lipid comprising PEG-DMG.

In some embodiments, a LNP comprises an ionizable amino lipid of any of Formula VI, VII or VIII, a phospholipid comprising DSPC, a structural lipid, and a PEG lipid comprising a compound having Formula XI.

In some embodiments, a LNP comprises an ionizable amino lipid of Formula VI, VII or VIII, a phospholipid comprising a compound having Formula VIII, a structural lipid, and the PEG lipid comprising a compound having Formula X or XI.

In some embodiments, a LNP comprises an ionizable amino lipid of Formula VI, VII or VIII, a phospholipid comprising a compound having Formula IX, a structural lipid, and the PEG lipid comprising a compound having Formula X or XI.

In some embodiments, a LNP comprises an ionizable amino lipid of Formula VI, VII or VIII, a phospholipid having Formula IX, a structural lipid, and a PEG lipid comprising a compound having Formula XI.

In some embodiments, the lipid nanoparticle comprises 49 mol % ionizable amino lipid, mol % DSPC, 38.5 mol % cholesterol, and 2.5 mol % DMG-PEG.

In some embodiments, the lipid nanoparticle comprises 49 mol % ionizable amino lipid, 11 mol % DSPC, 38.5 mol % cholesterol, and 1.5 mol % DMG-PEG.

In some embodiments, the lipid nanoparticle comprises 48 mol % ionizable amino lipid, 11 mol % DSPC, 38.5 mol % cholesterol, and 2.5 mol % DMG-PEG.

In some embodiments, a LNP comprises an N:P ratio of from about 2:1 to about 30:1.

In some embodiments, a LNP comprises an N:P ratio of about 6:1.

In some embodiments, a LNP comprises an N:P ratio of about 3:1, 4:1, or 5:1.

In some embodiments, a LNP comprises a wt/wt ratio of the ionizable amino lipid component to the RNA of from about 10:1 to about 100:1.

In some embodiments, a LNP comprises a wt/wt ratio of the ionizable amino lipid component to the RNA of about 20:1.

In some embodiments, a LNP comprises a wt/wt ratio of the ionizable amino lipid component to the RNA of about 10:1.

Some embodiments comprise a composition having one or more LNPs having a diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. Some embodiments comprise a composition having a mean LNP diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. In some embodiments, the composition has a mean LNP diameter from about 30 nm to about 150 nm, or a mean diameter from about 60 nm to about 120 nm.

A LNP may comprise or one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides.

In some embodiments, the composition comprises a liposome. A liposome is a lipid particle comprising lipids arranged into one or more concentric lipid bilayers around a central region. The central region of a liposome may comprises an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles, such as those described herein, may be used for a wide variety of applications, including the stealth delivery of therapeutic payloads with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., DNA molecules). The reduced stability may limit the broad applicability of the particulate carriers. As such, there remains a need for methods by which to improve the stability of nucleic acid (e.g., DNA) encapsulated within lipid nanoparticles.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, a LNP described herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

According to the disclosures herein, a lipid composition may comprise one or more lipids as described herein. Such lipids may include those useful in the preparation of lipid nanoparticle formulations as described above or as known in the art.

In some embodiments, a subject to which a composition comprising a nucleic acid and a lipid, is administered is a subject that suffers from or is at risk of suffering from a disease, disorder or condition, including a communicable or non-communicable disease, disorder or condition. As used herein, “treating” a subject can include either therapeutic use or prophylactic use relating to a disease, disorder or condition, and may be used to describe uses for the alleviation of symptoms of a disease, disorder or condition, uses for vaccination against a disease, disorder or condition, and uses for decreasing the contagiousness of a disease, disorder or condition, among other uses.

In some embodiments the nucleic acid is an DNA vaccine designed to achieve particular biologic effects. Exemplary vaccines feature DNA encoding a particular antigen of interest (or a DNA or DNAs encoding antigens of interest). In exemplary aspects, the vaccines feature a DNA or DNAs encoding antigen(s) derived from infectious diseases or cancers.

Diseases or conditions, in some embodiments include those caused by or associated with infectious agents, such as bacteria, viruses, fungi and parasites. Non-limiting examples of such infectious agents include Gram-negative bacteria, Gram-positive bacteria, RNA viruses (including (+)ssRNA viruses, (−)ssRNA viruses, dsRNA viruses), DNA viruses (including dsDNA viruses and ssDNA viruses), reverse transcriptase viruses (including ssRNA-RT viruses and dsDNA-RT viruses), protozoa, helminths, and ectoparasites.

Thus, the disclosure also encompasses infectious disease vaccines. The antigen of the infectious disease vaccine is a viral or bacterial antigen.

In some embodiments, a disease, disorder, or condition is caused by or associated with a virus.

The compositions are also useful for treating or preventing a symptom of diseases characterized by missing or aberrant protein activity, by replacing the missing protein activity or overcoming the aberrant protein activity. Multiple diseases are characterized by missing (or substantially diminished such that proper protein function does not occur) protein activity. Such proteins may not be present, are present in very low quantities or are essentially non-functional. Some aspects of the disclosure relate to a method for treating such conditions or diseases in a subject by introducing polynucleotide or cell-based therapeutics containing the alternative polynucleotides provided herein, wherein the alternative polynucleotides encode for a protein that replaces the protein activity missing from the target cells of the subject.

Diseases characterized by dysfunctional or aberrant protein activity include, but are not limited to, cancer and other proliferative diseases, genetic diseases (e.g., cystic fibrosis), autoimmune diseases, diabetes, neurodegenerative diseases, cardiovascular diseases, and metabolic diseases. Some aspects of the disclosure relate to a method for treating such conditions or diseases in a subject by introducing polynucleotide or cell-based therapeutics containing the polynucleotides provided herein, wherein the polynucleotides encode for a protein that antagonizes or otherwise overcomes the aberrant protein activity present in the cell of the subject.

In some embodiments, a composition disclosed herein does not comprise a pharmaceutical preservative. In other embodiments, a composition disclosed herein does comprise a pharmaceutical preservative. Non-limiting examples of pharmaceutical preservatives include methyl paragen, ethyl paraben, propyl paraben, butyl paraben, benzyl acohol, chlorobutanol, phenol, meta cresol (m-cresol), chloro cresol, benzoic acid, sorbic acid, thiomersal, phenylmercuric nitrate, bronopol, propylene glycol, benzylkonium chloride, and benzethionium chloride. In some embodiments, a composition disclosed herein does not comprise phenol, m-cresol, or benzyl alcohol. Compositions in which microbial growth is inhibited may be useful in the preparation of injectable formulations, including those intended for dispensing from multi-dose vials. Multi-dose vials refer to containers of pharmaceutical compositions from which multiple doses can be taken repeatedly from the same container. Compositions intended for dispensing from multi-dose vials typically must meet USP requirements for antimicrobial effectiveness.

In some embodiments, “administering” or “administration” means providing a material to a subject in a manner that is pharmacologically useful. In some embodiments, a composition disclosed herein is administered to a subject enterally. In some embodiments, an enteral administration of the composition is oral. In some embodiments, a composition disclosed herein is administered to the subject parenterally. In some embodiments, a composition disclosed herein is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs.

To “treat” a disease as the term is used herein, means to reduce the frequency or severity of at least one sign or symptom of a disease, disorder or condition experienced by a subject. The compositions described above or elsewhere herein are typically administered to a subject in an effective amount, that is, an amount capable of producing a desirable result. The desirable result will depend upon the active agent being administered. For example, an effective amount of a composition comprising a nucleic acid and a lipid may be an amount of the composition that is capable of increasing expression of a protein in the subject. A therapeutically acceptable amount may be an amount that is capable of treating a disease or condition, e.g., a disease or condition that that can be relieved by increasing expression of a protein in a subject. As is well known in the medical and veterinary arts, dosage for any one subject depends on many factors, including the subject's size, body surface area, age, the particular composition to be administered, the active ingredient(s) in the composition, the intended outcome of the administration, time and route of administration, general health, and other drugs being administered concurrently.

In some embodiments, a subject is administered a composition comprising a nucleic acid and a lipid I in an amount sufficient to increase expression of a protein in the subject.

In certain embodiments, LNP preparations (e.g., populations or formulations) are analyzed for polydispersity in size (e.g., particle diameter) and/or composition (e.g., amino lipid amount or concentration, phospholipid amount or concentration, structural lipid amount or concentration, PEG-lipid amount or concentration, DNA amount (e.g., mass) or concentration) and, optionally, further assayed for in vitro and/or in vivo activity. Fractions or pools thereof can also be analyzed for accessible DNA and/or purity (e.g., purity as determined by reverse-phase (RP) chromatography).

Particle size (e.g., particle diameter) can be determined by Dynamic Light Scattering (DLS). DLS measures a hydrodynamic diameter. Smaller particles diffuse more quickly, leading to faster fluctuations in the scattering intensity and shorter decay times for the autocorrelation function. Larger particles diffuse more slowly, leading to slower fluctuations in the scattering intensity and longer decay times in the autocorrelation function.

DNA purity can be determined by reverse phase high-performance liquid chromatography (RP-HPLC) size based separation. This method can be used to assess DNA integrity by a length-based gradient RP separation and UV detection of RNA at 260 nm. As used herein “main peak” or “main peak purity” refers to the RP-HPLC signal detected from DNA that corresponds to the full size DNA molecule loaded within a given LNP formulation. DNA purity can also be assessed by fragmentation analysis. Fragmentation analysis (FA) is a method by which nucleic acid (e.g., DNA) fragments can be analyzed by capillary electrophoresis. Fragmentation analysis involves sizing and quantifying nucleic acids (e.g., DNA), for example by using an intercalating dye coupled with an LED light source. Such analysis may be completed, for example, with a Fragment Analyzer from Advanced Analytical Technologies, Inc.

Compositions formed via the methods described herein may be particularly useful for administering an agent to a subject in need thereof. In some embodiments, the compositions are used to deliver a pharmaceutically active agent. In some instances, the compositions are used to deliver a prophylactic agent. The compositions may be administered in any way known in the art of drug delivery, for example, orally, parenterally, intravenously, intramuscularly, subcutaneously, intradermally, transdermally, intrathecally, submucosally, etc.

Once the compositions have been prepared, they may be combined with pharmaceutically acceptable excipients to form a pharmaceutical composition. As would be appreciated by one of skill in this art, the excipients may be chosen based on the route of administration as described below, the agent being delivered, and the time course of delivery of the agent.

Pharmaceutical compositions described herein and for use in accordance with the embodiments described herein may include a pharmaceutically acceptable excipient. As used herein, the term “pharmaceutically acceptable excipient” means a non-toxic, inert solid, semi-solid or liquid filler, diluent, encapsulating material or formulation auxiliary of any type. Some examples of materials which can serve as pharmaceutically acceptable excipients are sugars such as lactose, glucose, and sucrose; starches such as corn starch and potato starch; cellulose and its derivatives such as sodium carboxymethyl cellulose, methylcellulose, hydroxypropylmethylcellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions can be administered to humans and/or to animals, orally, parenterally, intracisternally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., the particles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, ethanol, U.S.P., and isotonic sodium chloride solution. In addition, sterile, fixed oils are conventionally employed as a solvent or suspending medium. For this purpose any bland fixed oil can be employed including synthetic mono or diglycerides. In addition, fatty acids such as oleic acid are used in the preparation of injectables.

The injectable formulations can be sterilized, for example, by filtration through a bacteria retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard filled gelatin capsules using such excipients as lactose or milk sugar as well as high molecular weight polyethylene glycols and the like.

The solid dosage forms of tablets, dragées, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also possible.

The ointments, pastes, creams, and gels may contain, in addition to the compositions, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the compositions, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compositions in a proper medium. Absorption enhancers can also be used to increase the flux of the compound across the skin. The rate can be controlled by either providing a rate controlling membrane or by dispersing the compositions in a polymer matrix or gel.

In other embodiments, the compositions are loaded and stored in prefilled syringes and cartridges for patient-friendly autoinjector and infusion pump devices.

Kits for use in preparing or administering the compositions are also provided. A kit for forming compositions may include any solvents, solutions, buffer agents, acids, bases, salts, targeting agent, etc. needed in the composition formation process. Different kits may be available for different targeting agents. In certain embodiments, the kit includes materials or reagents for purifying, sizing, and/or characterizing the resulting compositions. The kit may also include instructions on how to use the materials in the kit. The one or more agents (e.g., pharmaceutically active agent) to be contained within the composition are typically provided by the user of the kit.

Kits are also provided for using or administering the compositions. The compositions may be provided in convenient dosage units for administration to a subject. The kit may include multiple dosage units. For example, the kit may include 1-100 dosage units. In certain embodiments, the kit includes a week supply of dosage units, or a month supply of dosage units. In certain embodiments, the kit includes an even longer supply of dosage units. The kits may also include devices for administering the compositions. Exemplary devices include syringes, spoons, measuring devices, etc. The kit may optionally include instructions for administering the compositions (e.g., prescribing information).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As disclosed herein, the terms “composition” and “formulation” are used interchangeably.

### Sequence Identity

The terms “identity” and “sequence identity” refer to a relationship between the sequences of two or more polypeptides or polynucleotides (nucleic acids), as determined by comparing the sequences. Sequence identity also refers to the degree of sequence relatedness between or among sequences as determined by the number of matches between strings of two or more amino acid residues or nucleic acid residues. Sequence identity measures the percent of identical matches between the smaller of two or more sequences with gap alignments (if any) addressed by a particular mathematical model or computer program (e.g., “algorithms”). Sequence identity of related nucleic acids or proteins can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide have at least 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, promoters, and polynucleotides encoding peptides or polypeptides containing substitutions, insertions and/or additions, deletions and covalent modifications with respect to reference sequences are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### EXAMPLES

### Example 1: Strain Engineering for DNA Plasmid Replication

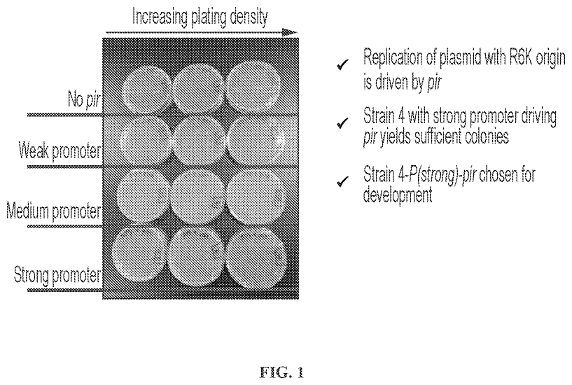

The γ origin of replication (from R6K) was used as the origin of replication for the DNA vector. Various pir expression cassettes were integrated into the genome of Strain 4, and their ability to replicate plasmid with a γ origin and a kanamycin resistance marker was determined. As shown in FIG. 1, the pir-containing strains were able to replicate the γ origin plasmid and produced colonies on LB+kanamycin agar plates (Note that the medium promoter strain was found to be corrupted and thus the data with respect to that strain was not considered). Due to robust growth and transformation efficiency with the γ origin-containing plasmid, the strain comprising the strong promoter construct was selected for further development in the production of markerless DNA.

### Example 2: Markerless DNA Strategy

A markerless plasmid DNA system has been developed that utilizes plasmid-borne expression of modified tRNA to suppress an amber mutation that had been introduced into an essential gene (see, e.g., Marie et al. J Gene Med. 2010. 12(4):323-32). Use of modified tRNA for amber, ochre, or opal suppression in a mutant bacterial strain enables positive selection of E. coli transformed with a plasmid, without the need for antibiotic resistance markers, which is an important consideration for developing DNA-based therapeutics for administration to a subject. Furthermore, tRNAs are generally quite small compared to ORFs that encode antibiotic resistance proteins. The amber mutation was introduced into a gene that is essential only in defined media. For plasmid DNA fermentation processes that are performed in rich or complex media, the markerless plasmid selection scheme should function in all media. This was an important problem with the prior art methods, as it is difficult to design a strain that is conditionally unable to grow in rich media but can also be rescued by expression of a tRNA.

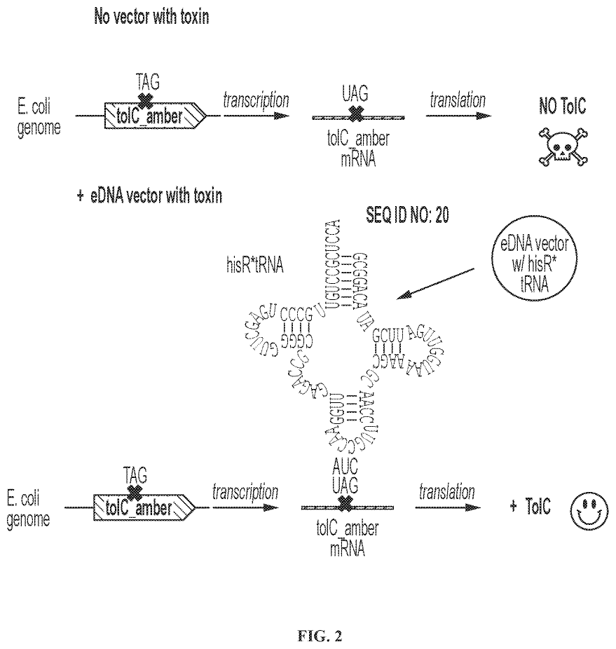

The TAG amber mutation was introduced into the tolC ORF a plasmid DNA production strain, Strain 4 (FIG. 2). TolC is the major outer membrane efflux pump component of E. coli. It is not an essential gene, but it is necessary for the efflux of many toxins, and a tolC knockout is lethal at elevated levels. The tolC mutant strain generated by the tolC::amber mutation was therefore hypersensitive to many toxins, such as low concentrations of nalidixic acid, irrespective of the growth media. While a tolC mutant has hypersensitivity to toxins, it can be cultured normally in rich media such as LB or Terrific Broth, which allows for the generation of competent cells that may be used for DNA synthesis in production.

### Example 3: Markerless Plasmid DNA

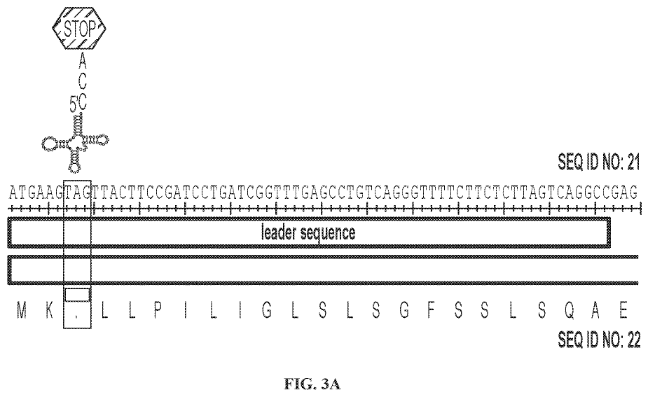

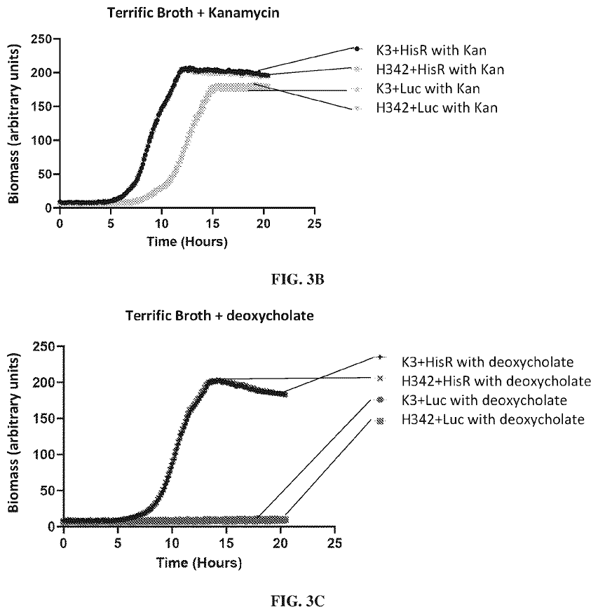

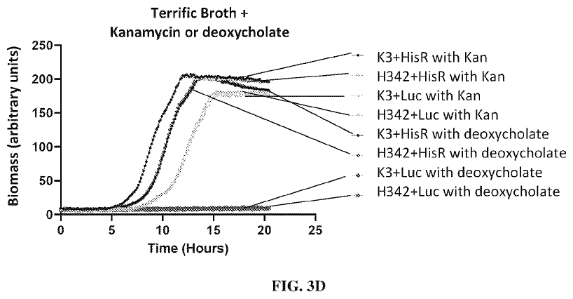

It was evaluated whether plasmid-borne expression of a mutant tRNA with an anticodon that is complementary to the tolC amber mutation would allow for expression of full-length TolC protein, and therefore allow the strain to grow in the presence of an otherwise lethal concentration of nalidixic acid (FIG. 2). In this case, a histidine tRNA, hisR, was modified with an anticodon loop that is complementary to the tolC amber STOP codon, UAG on RNA, which is encoded by TAG on DNA. Mutant E. coli strains were generated by introducing either a tolC::K3_amber (referred to as “K3”) or tolC::H342_amber (referred to as “H342”) amber mutation into the genome (FIG. 3A). Introduction of a STOP codon at an early codon of the open reading frame prevents the translation of non-functional TolC peptides with intact leader sequences, which would otherwise be translocated across the inner cell membrane into the periplasmic space or outer membrane. Each strain also contained codon-optimized tolC coding sequences. Both strains were transformed with a plasmid encoding either i) an amber suppressor tRNA that carries histidine (hisR), or ii) luciferase, as a control plasmid. Both plasmids carried a kanamycin-resistance marker to allow for plasmid maintenance during growth. These strains were then inoculated into LB media containing deoxycholate, and their growth was monitored @37° C. Each transformed strain was grown over 24 hours in TBAF++ media containing kanamycin (Kan, FIGS. 3B, 3D) or deoxycholate (DOC 150, FIGS. 3C-3D). All strains grew in the presence of kanamycin, though strains lacking the HisR tRNA grew more slowly than those transformed with the HisR-encoding plasmid (FIGS. 3B, 3D). However, the presence of DOC 150 was lethal to strains not expressing the HisR tRNA (FIGS. 3C-3D). These results indicate that deoxycholate allows for efficient positive selection of strains expressing the HisR tRNA.

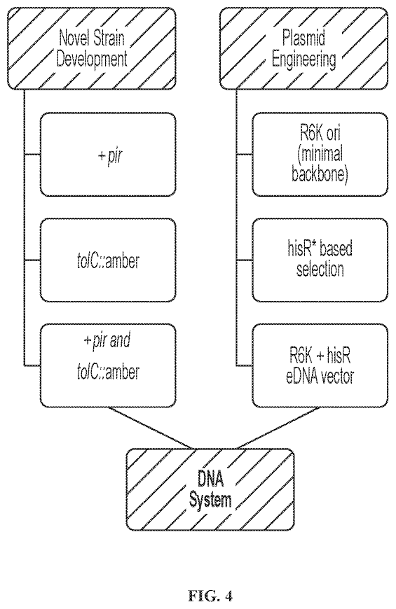

The combination of bacterial strain and plasmid provide a markerless DNA strain and vector platform for the production of markerless DNA (FIG. 4). The markerless DNA production platform is further improved by integrating the modifications described above into a single strain containing other modifications that improve plasmid production efficiency. These novel strains possess mutations that improve plasmid DNA cloning efficiency and plasmid stability, increase copy number, and reduce plasmid degradation during purification. The strain also expresses the pir gene to allow for replication of the DNA vector that carries the small γ origin of replication. Finally, the strains possess the tolC::amber mutations and, therefore, rely on the presence of the DNA vector comprising hisR* on the vector backbone.

### Example 4: Evaluation of Plasmid Productivity Using Markerless DNA

A plasmid encoding luciferase and a kanamycin resistance marker was modified to remove the sequence encoding the kanamycin resistance marker and insert a sequence encoding a modified hisR sequence, for suppression of a genomic amber mutation in tolC, which reduced the plasmid size by about 550 bp. E. coli strains containing a tolC::amber nonsense mutation were transformed with either the parent plasmid (Luc-KanR) or the modified plasmid (Luc-HisR). Each transformant was inoculated into shake flasks containing Terrific Broth with 200 ng/μL DOC or 50 μg/mL kanamycin for Luc-HisR and Luc-KanR strains, respectively, and incubated to begin a seed train. Seed cultures for each strain were transferred into Ambr250 bioreactors for fed-batch fermentations to evaluate plasmid and biomass yields for each strain. The bioreactors contained Terrific Broth and were run for over 120 hours, with broth containing 200 ng/μL DOC for fermentation of Luc-HisR-transformed strains to select for bacteria expressing the hisR tRNA, or 50 μg/mL kanamycin for fermentation of Luc-KanR-transformed strains.

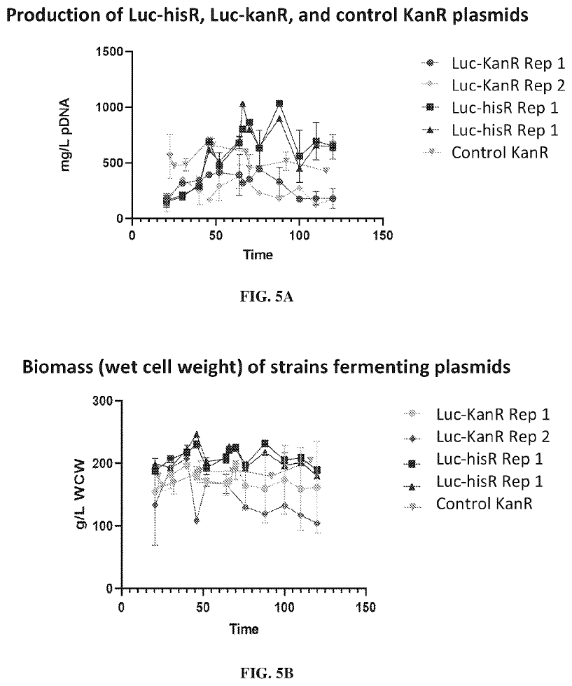

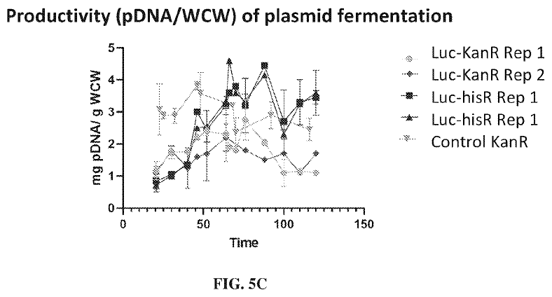

Samples were obtained at multiple times during fermentation to measure bacterial biomass and plasmid DNA production. The results of these experiments are shown in FIGS. 5A-5C, along with historical data from a previous fermentation experiment using a strain containing the Luc-KanR plasmid (“Control KanR”). Each strain reached comparable biomasses of around 200-220 g/L, but the Luc-HisR-transformed strain reached peak biomass earlier than the Luc-KanR-transformed strain, and this peak was higher for the Luc-HisR strain, indicating improved growth using amber suppressor hisR-based selection compared to kanamycin selection (FIG. 5B). Additionally, higher amounts of Luc-HisR plasmid were produced relative to Luc-KanR (FIG. 5A). Thus, the total productivity, in terms of plasmid DNA produced per unit bacterial biomass, was higher for bacteria producing the Luc-HisR plasmid than the Luc-KanR plasmid (FIG. 5C). These results demonstrate that the use of an amber suppressor tRNA (e.g., hisR) in place of an antibiotic (e.g., kanamycin) resistance marker markedly improves plasmid yield while also reducing total plasmid size. This approach also enables antibiotic-free selection of bacteria replicating markerless plasmids, reducing the metabolic burden (e.g., of expressing antibiotic resistance markers) on bacteria during fermentation.

### EXEMPLARY SEQUENCES

| SEQ | | |
|---|---|---|
| ID | | |
| NO | SEQUENCE | DESCRIPTION |
| 1 | CTGATGGCTAGCTCAGTCCTAGGGATTATGCTAGC | J23113 |
| | | promoter |
| 2 | TTTATGGCTAGCTCAGTCCTAGGTACAATGCTAGC | J23114 |
| | | promoter |
| 3 | TTTACGGCTAGCTCAGCCCTAGGTATTATGCTAGC | J23017 |
| | | promoter |
| 4 | TTGACATATGTATCCGCTCATGATATAATAACCC | Kan promoter |
| 5 | TGGTGCAAAACCTTTCGCGGTATGGCATGATAGCGCCCGGAAGAGAGTCAATTCAGG | LacIq promoter |
| 6 | TTGACAATTAATCATCCGGCTCGTATAATG | trc promoter |
| 7 | AAGTGCCTTCCCATCAAAAAAATATTCTCAACATAAAAAACTTTGTGTAATACTTGT | Lpp promoter |
| | AACG | |
| 8 | ATCTTGCTACCAGTGGAACAGCCACTAAGGATTCTGCAGTGAGAGCAGAGGGCCAGC | hAAT promoter |
| | TAAGTGGTACTCTCCCAGAGACTGTCTGACTCACGCCACCCCCTCCACCTTGGACAC | |
| | AGGACGCTGTGGTTTCTGAGCCAGGTACAATGACTCCTTTCGGTAAGTGCAGTGGAA | |
| | GCTGTACACTGCCCAGGCAAAGCGTCCGGGCAGCGTAGGCGGGCGACTCAGATCCCA | |
| | GCCAGTGGACTTAGCCCCTGTTTGCTCCTCCGATAACTGGGGTGACCTTGGTTAATA | |
| | TTCACCAGCAGCCTCCCCCGTTGCCCCTCTGGATCCACTGCTTAAATA | |
| 9 | ATGCGTTTGAAGGTGATGATGGATGTCAATAAGAAGACCAAGATCCGTCACCGCAAT | Nucleotide |
| | GAACTTAATCATACCCTTGCACAGTTGCCATTACCAGCGAAACGCGTCATGTATATG | sequence of pir |
| | GCCCTTGCCCCAATCGATAGTAAAGAGCCATTGGAGCGTGGGCGTGTGTTCAAAATC | (codon- |
| | CGCGCTGAGGATCTTGCAGCCTTAGCAAAGATCACCCCCTCATTAGCTTACCGTCAG | optimized for |
| | TTAAAGGAGGGTGGAAAACTGTTGGGAGCTTCAAAAATTTCGTTACGTGGAGACGAC | E. coli K12) |
| | ATCATCGCCCTTGCTAAGGAATTAAATTTACCTTTCACCGCGAAGAATTCTCCCGAA | |
| | GAGCTTGACCTTAATATCATTGAGTGGATTGCATACTCTCCGGATGAAGGTTACCTT | |
| | TCTTTGAAATTCACCCGCACGATCGAACCCTACATTAGTTCCCTGATCGGGAAGAAG | |
| | AATAAATTCACGACCCAGTTATTGACTGCATCCCTGCGCCTGTCCTCGCAATACTCC | |
| | AGTTCTCTGTACCAACTTATTCGTAAGCACTACTCAAACTTTAAGAAAAAAAACTAT | |
| | TTTATTATCAGTGTCGATGAGTTGAAGGAAGAGTTGATTGCGTACACCTTTGACAAG | |
| | GACGGAAATATCGAATACAAATATCCAGACTTCCCCATCTTTAAGCGTGATGTCCTG | |
| | AATAAAGCCATTGCAGAGATTAAAAAAAAAACGGAAATTTCATTCGTTGGATTTACA | |
| | GTCCACGAGAAAGAGGGCCGCAAAATCAGTAAATTGAAGTTTGAATTTGTTGTTGAT | |
| | GAAGACGAGTTTTCTGGGGATAAAGATGATGAAGCATTTTTTATGAACCTGAGCGAA | |
| | GCAGACGCAGCCTTTTTGAAGGTATTCGACGAAACGGTGCCGCCGAAAAAGGCAAAG | |
| | GGATAA | |
| 10 | MRLKVMMDVNKKTKIRHRNELNHTLAQLPLPAKRVMYMALAPIDSKEPLERGRVFKI | Amino acid |
| | RAEDLAALAKITPSLAYRQLKEGGKLLGASKISLRGDDIIALAKELNLPFTAKNSPE | sequence of Pi |
| | ELDLNIIEWIAYSPDEGYLSLKFTRTIEPYISSLIGKKNKFTTQLLTASLRLSSQYS | protein |
| | SSLYQLIRKHYSNFKKKNYFIISVDELKEELIAYTFDKDGNIEYKYPDFPIFKRDVL | (encoded by |
| | NKAIAEIKKKTEISFVGFTVHEKEGRKISKLKFEFVVDEDEFSGDKDDEAFFMNLSE | pir) |
| | ADAAFLKVFDETVPPKKAKG | |
| 11 | ATGAAGAAATTGCTCCCCATTCTTATCGGCCTGAGCCTTTCTGGGTTCAGTTCGTTG | Nucleic acid |
| | AGCCAGGCCGAGAACCTGATGCAAGTTTATCAGCAAGCACGCCTTAGTAACCCGGAA | sequence of |
| | TTGCGTAAGTCTGCCGCCGATCGTGATGCTGCCTTTGAAAAAATTAATGAAGCGCGC | wild-type tolC |
| | AGTCCATTACTGCCACAGCTAGGTTTAGGTGCAGATTACACCTATAGCAACGGCTAC | |
| | CGCGACGCGAACGGCATCAACTCTAACGCGACCAGTGCGTCCTTGCAGTTAACTCAA | |
| | TCCATTTTTGATATGTCGAAATGGCGTGCGTTAACGCTGCAGGAAAAAGCAGCAGGG | |
| | ATTCAGGACGTCACGTATCAGACCGATCAGCAAACCTTGATCCTCAACACCGCGACC | |
| | GCTTATTTCAACGTGTTGAATGCTATTGACGTTCTTTCCTATACACAGGCACAAAAA | |
| | GAAGCGATCTACCGTCAATTAGATCAAACCACCCAACGTTTTAACGTGGGCCTGGTA | |
| | GCGATCACCGACGTGCAGAACGCCCGCGCACAGTACGATACCGTGCTGGCGAACGAA | |
| | GTGACCGCACGTAATAACCTTGATAACGCGGTAGAGCAGCTGCGCCAGATCACCGGT | |
| | AACTACTATCCGGAACTGGCTGCGCTGAATGTCGAAAACTTTAAAACCGACAAACCA | |
| | CAGCCGGTTAACGCGCTGCTGAAAGAAGCCGAAAAACGCAACCTGTCGCTGTTACAG | |
| | GCACGCTTGAGCCAGGACCTGGCGCGCGAGCAAATTCGCCAGGCGCAGGATGGTCAC | |
| | TTACCGACTCTGGATTTAACGGCTTCTACCGGGATTTCTGACACCTCTTATAGCGGT | |
| | TCGAAAACCCGTGGTGCCGCTGGTACCCAGTATGACGATAGCAATATGGGCCAGAAC | |
| | AAAGTTGGCCTGAGCTTCTCGCTGCCGATTTATCAGGGCGGAATGGTTAACTCGCAG | |
| | GTGAAACAGGCACAGTACAACTTTGTCGGTGCCAGCGAGCAACTGGAAAGTGCCCAT | |
| | CGTAGCGTCGTGCAGACCGTGCGTTCCTCCTTCAACAACATTAATGCATCTATCAGT | |
| | AGCATTAACGCCTACAAACAAGCCGTAGTTTCCGCTCAAAGCTCATTAGACGCGATG | |
| | GAAGCGGGCTACTCGGTCGGTACGCGTACCATTGTTGATGTGTTGGATGCGACCACC | |
| | ACGTTGTACAACGCCAAGCAAGAGCTGGCGAATGCGCGTTATAACTACCTGATTAAT | |
| | CAGCTGAATATTAAGTCAGCTCTGGGTACGTTGAACGAGCAGGATCTGCTGGCACTG | |
| | AACAATGCGCTGAGCAAACCGGTTTCCACTAATCCGGAAAACGTTGCACCGCAAACG | |
| | CCGGAACAGAATGCTATTGCTGATGGTTATGCGCCTGATAGCCCGGCACCAGTCGTT | |
| | CAGCAAACATCCGCACGCACTACCACCAGTAACGGTCATAACCCTTTCCGTAACTGA | |
| 12 | MKKLLPILIGLSLSGFSSLSQAENLMQVYQQARLSNPELRKSAADRDAAFEKINEAR | Amino acid |
| | SPLLPQLGLGADYTYSNGYRDANGINSNATSASLQLTQSIFDMSKWRALTLQEKAAG | sequence of |
| | IQDVTYQTDQQTLILNTATAYFNVLNAIDVLSYTQAQKEAIYRQLDQTTQRENVGLV | wild-type TolC |
| | AITDVQNARAQYDTVLANEVTARNNLDNAVEQLRQITGNYYPELAALNVENFKTDKP | (signal peptide |
| | QPVNALLKEAEKRNLSLLQARLSQDLAREQIRQAQDGHLPTLDLTASTGISDTSYSG | underlined) |
| | SKTRGAAGTQYDDSNMGQNKVGLSFSLPIYQGGMVNSQVKQAQYNFVGASEQLESAH | |
| | RSVVQTVRSSENNINASISSINAYKQAVVSAQSSLDAMEAGYSVGTRTIVDVLDATT | |
| | TLYNAKQELANARYNYLINQLNIKSALGTLNEQDLLALNNALSKPVSINPENVAPQT | |
| | PEQNAIADGYAPDSPAPVVQQTSARTTTSNGHNPERN | |
| 13 | GGTGGCTATAGCTCAGTTGGTAGAGCCCTGGATTCTAAATCCAGTTGTCGTGGGTTC | HisR amber |
| | GAATCCCATTAGCCACCCCA | suppressor |
| | | tRNA |
| | | (mutations |
| | | underlined) |
| 14 | GGTGGCTATAGCTCAGTTGGTAGAGCCCTGGATTTTAAATCCAGTTGTCGTGGGTTC | HisR ochre |
| | GAATCCCATTAGCCACCCCA | suppressor |
| | | tRNA |
| | | (mutations |
| | | underlined) |
| 15 | GGTGGCTATAGCTCAGTTGGTAGAGCCCTGGATTTCAAATCCAGTTGTCGTGGGTTC | HisR opal |
| | GAATCCCATTAGCCACCCCA | suppressor |
| | | tRNA |
| | | (mutations |
| | | underlined) |
| 16 | TCTTACTTCTTTGCGTAGCTGTTAAATACAGCGTTGTTTTGATAAAATCATCATTAT | R6K alpha |
| | CATCGATAATGCTTTCTTCAATTTTTTTATCCTTACTCTTTAATAAAGCACTTGCTA | (R6Kα) origin |
| | ATAACTTCATACCTTTTGCAACTGTCAAATTTGGTTCATCAGGGTAAATGCTTTTAA | of replication |
| | GGCATACTAACAAATAATCATGGTCTTCATCTTCAACTCTAAACTGAATTTTTTTCA | |
| | TCATAACTCCCAACAAGAACCGACTGTAGGTCACCGGGCAAACGCTGAAAAATAACG | |
| | TCGAATGACGTCATTTTGCGGCGTTTGCCCTATCCTGCATCGCAGTAGAAAATGCCA | |
| | CAACTGAAATTGTGCTTCAGTATGTACAGAAATGCAAAATCTGAGGGATTTCGTAGC | |
| | TGAAAGATCGCCAGTCTTCGACCGTAAGGATAGGAGTTGCTGTAAGACCTGTGCGGG | |
| | GCTGTTCGCTTCGCGAACGGGTCTGGCAGGGGGCGCAAGCGCTGTGCTGTGATATAT | |
| | GCAAAAGAAGCACCTCCCGCAAACGGG | |
| 17 | AGATCTATGGATGTTGAGAATGTAGTATGGTTATACTGCGATGCAGGATAGGGCAAA | R6K beta |
| | CGCCGTAAAATGACGTCTTTGACGTTATTTTTCAGCGCTTGCCCGGTGACCTACAGT | (R6Kβ) origin |
| | CGGTTCTTGTTGGGAGATTTTATGAAGTTTATAGTAAAGGATTTTATCAGTGATAAA | of replication |
| | TATGCAAAGGCTATTAACATTTTAAATGATAACCTTAAAGAAAACTACTATGTTTTT | |
| | TATGGTGTAAGGTTAAGTGAAATTCTTTTTCCTGCAAGTGATTATGGTACAGATGAT | |
| | TTTTTTAAGGAGTTTGAGGAAATAAACAACGTTACCTTGCCTTTAGTTGTTTTTGAA | |
| | ATAAATGAACGTGAACCTGTGATTGTAATTGGTTTTGATGAAATAAATGGTGCGATT | |
| | CTTATAGAGAAATCCGGTATAAAGGTTTTAGTAATCGATAATCTATCGGCGTTGCTG | |
| | ACCAATGAAACATTGGGTGTCTTCTTCAAGTAACAGATCTAGCTGAAAATCGTCATC | |
| | AAAACTGTGCACCTTATCTG | |
| 18 | AAGCTTTAAAAGCCTTATATATTCTTTTTTTTCTTATAAAACTTAAAACCTTAGAGG | R6K gamma |
| | CTATTTAAGTTGCTGATTTATATTAATTTTATTGTTCAAACATGAGAGCTTAGTACG | (R6Kγ) origin |
| | TGAAACATGAGAGCTTAGTACGTTAGCCATGAGAGCTTAGTACGTTAGCCATGAGGG | of replication |
| | TTTAGTTCGTTAAACATGAGAGCTTAGTACGTTAAACATGAGAGCTTAGTACGTGAA | |
| | ACATGAGAGCTTAGTACGTACTATCAACAGGTTGAACTGCTGATCTTCAGATCT | |
| 19 | ATCCCTGGCTTGTTGTCCACAACCGTTAAACCTTAAAAGCTTTAAAAGCCTTATATA | HisR vector |
| | TTCTTTTTTTTCTTATAAAACTTAAAACCTTAGAGGCTATTTAAGTTGCTGATTTAT | with R6Kγ |
| | ATTAATTTTATTGTTCAAACATGAGAGCTTAGTACGTGAAACATGAGAGCTTAGTAC | origin of |
| | GTTAGCCATGAGAGCTTAGTACGTTAGCCATGAGGGTTTAGTTCGTTAAACATGAGA | replication |
| | GCTTAGTACGTTAAACATGAGAGCTTAGTACGTGAAACATGAGAGCTTAGTACGTAC | |
| | TATCAACAGGTTGAACTGCGGATCTTGAAGTGCCTTCCCATCAAAAAAATATTCTCA | |
| | ACATAAAAAACTTTGTGTAATACTTGTAACGGGTGGCTATAGCTCAGTTGGTAGAGC | |
| | CCTGGATTCTAAATCCAGTTGTCGTGGGTTCGAATCCCATTAGCCACCCCACCCGGG | |
| | CGCCGAAAACCCCGCTTCGGCGGGGTTTTGCCGC | |

What is claimed is:

1. A genetically modified microorganism comprising
(i) a genome comprising a nonsense mutation in a gene encoding an efflux pump or import protein; and
(ii) a vector comprising a nucleic acid sequence encoding a suppressor tRNA, wherein the nonsense mutation comprises a first STOP codon, and the suppressor tRNA comprises an anticodon that is complementary to the first STOP codon.

2. The genetically modified microorganism of claim 1, wherein the gene encoding an efflux pump or import protein comprises a second STOP codon that is downstream of the first STOP codon, wherein the second STOP codon comprises a nucleic acid sequence that is not the nucleic acid sequence of the first STOP codon.

3. The genetically modified microorganism of claim 2, wherein the first STOP codon comprises the nucleic acid sequence TAG or UAG.

4. The genetically modified microorganism of any one of claims 1-3, wherein the first STOP codon comprises the nucleic acid sequence TAA or UAA.

5. The genetically modified microorganism of any one of claims 1-4, wherein the first STOP codon comprises the nucleic acid sequence TGA or UGA.

6. The genetically modified microorganism of any one of claims 1-5, wherein the first STOP codon is located in the first 400, first 300, first 250, first 200, first 150, first 100, first 90, first 80, first 70, first 60, first 50, first 40, first 30, first 20, first 10, or first 5 codons of an open reading frame in the gene encoding the efflux pump or import protein.

7. The genetically modified microorganism of any one of claims 1-6, wherein the first STOP codon is located in the first 10 codons of an open reading frame in the gene encoding the efflux pump or import protein.

8. The genetically modified microorganism of any one of claims 1-7, wherein the suppressor tRNA is a histidine tRNA.

9. The genetically modified microorganism of any one of claims 1-8, wherein the genome comprises a nucleic acid sequence encoding pir.

10. The genetically modified microorganism of claim 9, wherein a first promoter is operably linked to the nucleic acid sequence encoding pir.

11. The genetically modified microorganism of claim 10, wherein the first promoter is selected from the group consisting of a Kan promoter, LacIq promoter, trc promoter, Lpp promoter, and J23107 promoter.

12. The genetically modified microorganism of claim 10 or claim 11, wherein the first promoter comprises a nucleic acid sequence with at least 90% sequence identity to the nucleic acid sequence of any one of SEQ ID NOs: 3-7.

13. The genetically modified microorganism of any one of claims 1-12, wherein the vector comprises an R⁶Kγ origin of replication.

14. The genetically modified microorganism of any one of claims 1-13, wherein the vector comprises fewer than 1,000, fewer than 900, fewer than 800, fewer than 700, fewer than 600, or fewer than 500 nucleotides.

15. The genetically modified microorganism of any one of claims 1-14, wherein the genome is an E. coli genome.

16. The genetically modified microorganism of any one of claims 1-15, wherein the genome comprises a nonsense mutation in a gene encoding an efflux pump, wherein the gene encoding an efflux pump is acrAB, acrD, acrEF, emrD, emrE, emrKY, mdfA, tehAB, tolC, ybhGFSR, ybjY, ybjZ, yegM, yegNO, yhiUV, yjcP, ylcB, or yohG.

17. The genetically modified microorganism of claim 16, wherein the gene encoding an efflux pump is tolC.

18. The genetically modified microorganism of any one of claims 1-17, wherein the microorganism is capable of growing in the presence of a selective agent.

19. The genetically modified microorganism of claim 18, wherein the selective agent is ampicillin, chloramphenicol, florfenicol, clotrimazole, puromycin, erythromycin, methotrexate, novobiocin, ciprofloxacin, norfloxacin, nalidixic acid, rifampin, fusidic acid, streptomycin, sulfacetamide, tetracycline, deoxycholate, sodium cholate, sodium taurodeoxycholate, sodium oxalate, proflavine, crystal violet, acriflavin, ethidium bromide, tetraphenylphosphonium, rhodamine 6G, tetraphenylarsonium chloride, dequalinium chloride, benzalkonium chloride, daunomycin, plumbagin, or methyl viologen.

20. The genetically modified microorganism of claim 19, wherein the selective agent is nalidixic acid.

21. The genetically modified microorganism of claim 19, wherein the selective agent is deoxycholate.

22. The genetically modified microorganism of any one of claims 1-21, wherein the genome comprises a nonsense mutation in a gene encoding an import protein.

23. The genetically modified microorganism of claim 22, wherein the import protein is selected from the group consisting of mppA and oppBCDF.

24. The genetically modified microorganism of claim 22 or 23, wherein the microorganism is an auxotroph, wherein the auxotroph is not capable of synthesizing one or more nutrients.

25. The genetically modified microorganism of claim 24, wherein the auxotroph is not capable of synthesizing one or more amino acids.

26. A method of enriching a population of microorganisms for the genetically modified microorganism of any one of claims 16-21, the method comprising exposing a population of microorganisms to a selective agent, wherein the frequency of the genetically modified microorganism in the population is increased after exposure to the selective agent.

27. The method of claim 26, wherein the selective agent is ampicillin, chloramphenicol, florfenicol, clotrimazole, puromycin, erythromycin, methotrexate, novobiocin, ciprofloxacin, norfloxacin, nalidixic acid, rifampin, fusidic acid, streptomycin, sulfacetamide, tetracycline, deoxycholate, sodium cholate, sodium taurodeoxycholate, sodium oxalate, proflavine, crystal violet, acriflavine, ethidium bromide, tetraphenylphosphonium, rhodamine 6G, tetraphenylarsonium chloride, dequalinium chloride, benzalkonium chloride, daunomycin, plumbagin, or methyl viologen.

28. The method of claim 27, wherein the selective agent is nalidixic acid.

29. The method of claim 27, wherein the selective agent is deoxycholate.

30. A method of enriching a population of microorganisms for the genetically modified microorganism of any one of claims 22-25, the method comprising growing a population of microorganisms in the presence of a nutrient, wherein the frequency of the genetically modified microorganism in the population is increased after growth in the presence of the nutrient.

31. The method of claim 30, wherein the nutrient is an amino acid.

32. A method of producing a markerless DNA, the method comprising
(i) culturing the genetically modified microorganism of any one of claims 1-25 under conditions suitable for replication of the vector; and
(ii) isolating the vector from the microorganism to obtain a markerless DNA.

33. A markerless DNA produced by the method of claim 32.

34. The markerless DNA of claim 33, further comprising an open reading frame encoding a protein.

35. The markerless DNA of claim 34, further comprising a second promoter, wherein the second promoter is operably linked to the open reading frame encoding the protein.

36. The markerless DNA of claim 35, wherein the open reading frame is codon optimized for expression in a cell.

37. The markerless DNA of claim 36, wherein the open reading frame is codon optimized for expression in a human cell.

38. A markerless DNA comprising
(i) an origin of replication;
(ii) a nucleic acid sequence encoding a suppressor tRNA; and
(iii) a nucleic acid sequence with at least 90% sequence identity to SEQ ID NO: 19. wherein the suppressor tRNA comprises an anticodon that is complementary to a STOP codon.

39. The markerless DNA of claim 38, wherein the origin of replication comprises fewer than 600, fewer than 500, fewer than 400, or fewer than 350 nucleotides.

40. The markerless DNA of claim 39, wherein the origin of replication is an R⁶Kγ origin of replication.

41. The markerless DNA of any one of claims 38-40, wherein the STOP codon comprises the nucleic acid sequence TAG or UAG.

42. The markerless DNA of any one of claims 38-40, wherein the STOP codon comprises the nucleic acid sequence TAA or UAA.

43. The markerless DNA of any one of claims 38-40, wherein the STOP codon comprises the nucleic acid sequence TGA or UGA.

44. The markerless DNA of any one of claims 38-43, wherein the suppressor tRNA is a histidine tRNA.

45. The markerless DNA of any one of claims 38-44, further comprising an open reading frame encoding a protein.

46. The markerless DNA of claim 45, further comprising a promoter, wherein the promoter is operably linked to the open reading frame encoding the protein.

47. The markerless DNA of claim 45 or 46, wherein the open reading frame is codon optimized for expression in a cell.

48. The markerless DNA of claim 47, wherein the open reading frame is codon optimized for expression in a human cell.

49. The markerless DNA of any one of claims 38-48, wherein the markerless DNA encodes an mRNA.

50. A composition comprising the markerless DNA of any one of claims 38-49, wherein the markerless DNA is formulated in a lipid nanoparticle.

51. The composition of claim 50, wherein the lipid nanoparticle comprises an ionizable lipid, a neutral lipid, a sterol, and a polyethylene glycol (PEG)-modified lipid.

52. The composition of claim 50 or claim 51, wherein the lipid nanoparticle comprises: 40-55 mol % ionizable amino lipid; 5-15 mol % non-cationic lipid; 35-45 mol % sterol; and 1-5 mol % PEG-modified lipid.

53. A pharmaceutical composition comprising the markerless DNA of any one of claims 38-49, or the composition of any one of claims 50-52.

54. The pharmaceutical composition of claim 53, further comprising a pharmaceutically acceptable excipient.

55. A method comprising administering the markerless DNA of any one of claims 38-49, the composition of any one of claims 50-52, or the pharmaceutical composition of claim 53 or claim 54 to a subject in need thereof.

56. A method for delivering a markerless DNA to a specific cell type in a subject in need thereof, comprising administering the markerless DNA of any one of claims 38-49, the composition of any one of claims 50-52, or the pharmaceutical composition of claim 53 or claim 54 to a subject in need thereof.

57. A method of treating a disease or condition in a subject in need thereof, comprising administering the markerless DNA of any one of claims 38-49, the composition of any one of claims 50-52, or the pharmaceutical composition of claim 53 or claim 54 to the subject.

58. The method of any one of claims 55-57, wherein the subject is a mammal.

59. The method of any one of claims 55-58, wherein the subject is a human.


SEQUENCE LISTING (22 sequences)

SEQ ID NO: 1 - DNA, 35 bp, synthetic construct
  misc_feature 1..35: note = Synthetic
  source 1..35: mol_type = other DNA
ctgatggctagctcagtcctagggattatgctagc

SEQ ID NO: 2 - DNA, 35 bp, synthetic construct
  misc_feature 1..35: note = Synthetic
  source 1..35: mol_type = other DNA
tttatggctagctcagtcctaggtacaatgctagc

SEQ ID NO: 3 - DNA, 35 bp, synthetic construct
  misc_feature 1..35: note = Synthetic
  source 1..35: mol_type = other DNA
tttacggctagctcagccctaggtattatgctagc

SEQ ID NO: 4 - DNA, 34 bp, synthetic construct
  misc_feature 1..34: note = Synthetic
  source 1..34: mol_type = other DNA
ttgacatatgtatccgctcatgatataataaccc

SEQ ID NO: 5 - DNA, 57 bp, synthetic construct
  misc_feature 1..57: note = Synthetic
  source 1..57: mol_type = other DNA
tggtgcaaaacctttcgcggtatggcatgatagcgcccggaagagagtcaattcagg

SEQ ID NO: 6 - DNA, 30 bp, synthetic construct
  misc_feature 1..30: note = Synthetic
  source 1..30: mol_type = other DNA
ttgacaattaatcatccggctcgtataatg

SEQ ID NO: 7 - DNA, 61 bp, synthetic construct
  misc_feature 1..61: note = Synthetic
  source 1..61: mol_type = other DNA
aagtgccttcccatcaaaaaaatattctcaacataaaaaactttgtgtaatacttgtaacg

SEQ ID NO: 8 - DNA, 333 bp, synthetic construct
  misc_feature 1..333: note = Synthetic
  source 1..333: mol_type = other DNA
atcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaata

SEQ ID NO: 9 - DNA, 918 bp, synthetic construct
  misc_feature 1..918: note = Synthetic
  source 1..918: mol_type = other DNA
atgcgtttgaaggtgatgatggatgtcaataagaagaccaagatccgtcaccgcaatgaacttaatcatacccttgcacagttgccattaccagcgaaacgcgtcatgtatatggcccttgccccaatcgatagtaaagagccattggagcgtgggcgtgtgttcaaaatccgcgctgaggatcttgcagccttagcaaagatcaccccctcattagcttaccgtcagttaaaggagggtggaaaactgttgggagcttcaaaaatttcgttacgtggagacgacatcatcgcccttgctaaggaattaaatttacctttcaccgcgaagaattctcccgaagagcttgaccttaatatcattgagtggattgcatactctccggatgaaggttacctttctttgaaattcacccgcacgatcgaaccctacattagttccctgatcgggaagaagaataaattcacgacccagttattgactgcatccctgcgcctgtcctcgcaatactccagttctctgtaccaacttattcgtaagcactactcaaactttaagaaaaaaaactattttattatcagtgtcgatgagttgaaggaagagttgattgcgtacacctttgacaaggacggaaatatcgaatacaaatatccagacttccccatctttaagcgtgatgtcctgaataaagccattgcagagattaaaaaaaaaacggaaatttcattcgttggatttacagtccacgagaaagagggccgcaaaatcagtaaattgaagtttgaatttgttgttgatgaagacgagttttctggggataaagatgatgaagcattttttatgaacctgagcgaagcagacgcagcctttttgaaggtattcgacgaaacggtgccgccgaaaaaggcaaagggataa

SEQ ID NO: 10 - protein, 305 aa, synthetic construct
  REGION 1..305: note = Synthetic
MRLKVMMDVNKKTKIRHRNELNHTLAQLPLPAKRVMYMALAPIDSKEPLERGRVFKIRAEDLAALAKITPSLAYRQLKEGGKLLGASKISLRGDDIIALAKELNLPFTAKNSPEELDLNIIEWIAYSPDEGYLSLKFTRTIEPYISSLIGKKNKFTTQLLTASLRLSSQYSSSLYQLIRKHYSNFKKKNYFIISVDELKEELIAYTFDKDGNIEYKYPDFPIFKRDVLNKAIAEIKKKTEISFVGFTVHEKEGRKISKLKFEFVVDEDEFSGDKDDEAFFMNLSEADAAFLKVFDETVPPKKAKG

SEQ ID NO: 11 - DNA, 1482 bp, synthetic construct
  misc_feature 1..1482: note = Synthetic
  source 1..1482: mol_type = other DNA
atgaagaaattgctccccattcttatcggcctgagcctttctgggttcagttcgttgagccaggccgagaacctgatgcaagtttatcagcaagcacgccttagtaacccggaattgcgtaagtctgccgccgatcgtgatgctgcctttgaaaaaattaatgaagcgcgcagtccattactgccacagctaggtttaggtgcagattacacctatagcaacggctaccgcgacgcgaacggcatcaactctaacgcgaccagtgcgtccttgcagttaactcaatccatttttgatatgtcgaaatggcgtgcgttaacgctgcaggaaaaagcagcagggattcaggacgtcacgtatcagaccgatcagcaaaccttgatcctcaacaccgcgaccgcttatttcaacgtgttgaatgctattgacgttctttcctatacacaggcacaaaaagaagcgatctaccgtcaattagatcaaaccacccaacgttttaacgtgggcctggtagcgatcaccgacgtgcagaacgcccgcgcacagtacgataccgtgctggcgaacgaagtgaccgcacgtaataaccttgataacgcggtagagcagctgcgccagatcaccggtaactactatccggaactggctgcgctgaatgtcgaaaactttaaaaccgacaaaccacagccggttaacgcgctgctgaaagaagccgaaaaacgcaacctgtcgctgttacaggcacgcttgagccaggacctggcgcgcgagcaaattcgccaggcgcaggatggtcacttaccgactctggatttaacggcttctaccgggatttctgacacctcttatagcggttcgaaaacccgtggtgccgctggtacccagtatgacgatagcaatatgggccagaacaaagttggcctgagcttctcgctgccgatttatcagggcggaatggttaactcgcaggtgaaacaggcacagtacaactttgtcggtgccagcgagcaactggaaagtgcccatcgtagcgtcgtgcagaccgtgcgttcctccttcaacaacattaatgcatctatcagtagcattaacgcctacaaacaagccgtagtttccgctcaaagctcattagacgcgatggaagcgggctactcggtcggtacgcgtaccattgttgatgtgttggatgcgaccaccacgttgtacaacgccaagcaagagctggcgaatgcgcgttataactacctgattaatcagctgaatattaagtcagctctgggtacgttgaacgagcaggatctgctggcactgaacaatgcgctgagcaaaccggtttccactaatccggaaaacgttgcaccgcaaacgccggaacagaatgctattgctgatggttatgcgcctgatagcccggcaccagtcgttcagcaaacatccgcacgcactaccaccagtaacggtcataaccctttccgtaactga

SEQ ID NO: 12 - protein, 493 aa, synthetic construct
  REGION 1..493: note = Synthetic
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 13 - DNA, 77 bp, synthetic construct
  misc_feature 1..77: note = Synthetic
  source 1..77: mol_type = other DNA
ggtggctatagctcagttggtagagccctggattctaaatccagttgtcgtgggttcgaatcccattagccacccca

SEQ ID NO: 14 - DNA, 77 bp, synthetic construct
  misc_feature 1..77: note = Synthetic
  source 1..77: mol_type = other DNA
ggtggctatagctcagttggtagagccctggattttaaatccagttgtcgtgggttcgaatcccattagccacccca

SEQ ID NO: 15 - DNA, 77 bp, synthetic construct
  misc_feature 1..77: note = Synthetic
  source 1..77: mol_type = other DNA
ggtggctatagctcagttggtagagccctggatttcaaatccagttgtcgtgggttcgaatcccattagccacccca

SEQ ID NO: 16 - DNA, 540 bp, synthetic construct
  misc_feature 1..540: note = Synthetic
  source 1..540: mol_type = other DNA
tcttacttctttgcgtagctgttaaatacagcgttgttttgataaaatcatcattatcatcgataatgctttcttcaatttttttatccttactctttaataaagcacttgctaataacttcataccttttgcaactgtcaaatttggttcatcagggtaaatgcttttaaggcatactaacaaataatcatggtcttcatcttcaactctaaactgaatttttttcatcataactcccaacaagaaccgactgtaggtcaccgggcaaacgctgaaaaataacgtcgaatgacgtcattttgcggcgtttgccctatcctgcatcgcagtagaaaatgccacaactgaaattgtgcttcagtatgtacagaaatgcaaaatctgagggatttcgtagctgaaagatcgccagtcttcgaccgtaaggataggagttgctgtaagacctgtgcggggctgttcgcttcgcgaacgggtctggcagggggcgcaagcgctgtgctgtgatatatgcaaaagaagcacctcccgcaaacggg

SEQ ID NO: 17 - DNA, 533 bp, synthetic construct
  misc_feature 1..533: note = Synthetic
  source 1..533: mol_type = other DNA
agatctatggatgttgagaatgtagtatggttatactgcgatgcaggatagggcaaacgccgtaaaatgacgtctttgacgttatttttcagcgcttgcccggtgacctacagtcggttcttgttgggagattttatgaagtttatagtaaaggattttatcagtgataaatatgcaaaggctattaacattttaaatgataaccttaaagaaaactactatgttttttatggtgtaaggttaagtgaaattctttttcctgcaagtgattatggtacagatgatttttttaaggagtttgaggaaataaacaacgttaccttgcctttagttgtttttgaaataaatgaacgtgaacctgtgattgtaattggttttgatgaaataaatggtgcgattcttatagagaaatccggtataaaggttttagtaatcgataatctatcggcgttgctgaccaatgaaacattgggtgtcttcttcaagtaacagatctagctgaaaatcgtcatcaaaactgtgcaccttatctg

SEQ ID NO: 18 - DNA, 282 bp, synthetic construct
  misc_feature 1..282: note = Synthetic
  source 1..282: mol_type = other DNA
aagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatct

SEQ ID NO: 19 - DNA, 490 bp, synthetic construct
  misc_feature 1..490: note = Synthetic
  source 1..490: mol_type = other DNA
atccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgcggatcttgaagtgccttcccatcaaaaaaatattctcaacataaaaaactttgtgtaatacttgtaacgggtggctatagctcagttggtagagccctggattctaaatccagttgtcgtgggttcgaatcccattagccaccccacccgggcgccgaaaaccccgcttcggcggggttttgccgc

SEQ ID NO: 20 - RNA, 78 nt, synthetic construct
  misc_feature 1..78: note = Synthetic
  source 1..78: mol_type = other RNA
gcggacatagcttagttggtaaaagcgcaaccttgccaaggttgagaccgcggggttcgagtcccgttgtccgctcca

SEQ ID NO: 21 - DNA, 69 bp, synthetic construct
  misc_feature 1..69: note = Synthetic
  source 1..69: mol_type = other DNA
atgaagtagttacttccgatcctgatcggtttgagcctgtcagggttttcttctcttagtcaggccgag

SEQ ID NO: 22 - protein, 20 aa, synthetic construct
  REGION 1..20: note = Synthetic
LLPILIGLSLSGFSSLSQAE